SEQUENCE LISTING (22 sequences)

SEQ ID NO: 1 - DNA, 1894 bp, Homo sapiens
agtcgacccacgcgtccgcctcacacgactgtgatccgattctttccagcggcttctgcaaccaagcgggtcttacccccggtcctccgcgtctccagtcctcgcacctggaaccccaacgtccccgagagtccccgaatccccgctcccaggctacctaagaggatgagcggtgctccgacggccggggcagccctgatgctctgcgccgccaccgccgtgctactgagcgctcagggcggacccgtgcagtccaagtcgccgcgctttgcgtcctgggacgagatgaatgtcctggcgcacggactcctgcagctcggccaggggctgcgcgaacacgcggagcgcacccgcagtcagctgagcgcgctggagcggcgcctgagcgcgtgcgggtccgcctgtcagggaaccgagggtccaccgaccctcccgttagcccctgagagccgggtggaccctgaggtccttcacagcctgcagacacaactcaaggctcagaacagcaggatccagcaactcttccacaaggtggcccagcagcagcggcacctggagaagcagcacctgcgaattcagcatctgcaaagccagtttggcctcctggaccacaagcacctagaccatgaggtggccaagcctgcccgaagaaagaggctgcccgagatggcccagccagttgacccggctcacaatgtcagccgcctgcaccggctgcccagggattgccaggagctgttccaggttggggagaggcagagtggactatttgaaatccagcctcaggggtctccgccatttttggtgaactgcaagatgacctcagatggaggctggacagtaattcagaggcgccacgatggctcagtggacttcaaccggccctgggaagcctacaaggcggggtttggggatccccacggcgagttctggctgggtctggagaaggtgcatagcatcacgggggaccgcaacagccgcctggccgtgcagctgcgggactgggatggcaacgccgagttgctgcagttctccgtgcacctgggtggcgaggacacggcctatagcctgcagctcactgcacccgtggccggccagctgggcgccaccaccgtcccacccagcggcctctccgtacccttctccacttgggaccaggatcacgacctccgcagggacaagaactgcgccaagagcctctctggaggctggtggtttggcacctgcagccattccaacctcaacggccagtacttccgctccatcccacagcagcggcagaagcttaagaagggaatcttctggaagacctggcggggccgctactacccgctgcaggccaccaccatgttgatccagcccatggcagcagaggcagcctcctagcgtcctggctgggcctggtcccaggcccacgaaagacggtgactcttggctctgcccgaggatgtggccgttccctgcctgggcaggggctccaaggaggggccatctggaaacttgtggacagagaagaagaccacgactggagaagccccctttctgagtgcaggggggctgcatgcgttgcctcctgagatcgaggctgcaggatatgctcagactctagaggcgtggaccaaggggcatggagcttcactccttgctggccagggagttggggactcagagggaccacttggggccagccagactggcctcaatggcggactcagtcacattgactgacggggaccagggcttgtgtgggtcgagagcgccctcatggtgctggtgctgttgtgtgtaggtcccctggggacacaagcaggcgccaatggtatctgggcggagctcacagagttcttggaataaaagcaacctcagaacaaaaaaaaaaaaaaagggcggccgc

SEQ ID NO: 2 - protein, 406 aa, Homo sapiens
MSGAPTAGAALMLCAATAVLLSAQGGPVQSKSPRFASWDEMNVLAHGLLQLGQGLREHAERTRSQLSALERRLSACGSACQGTEGPPTLPLAPESRVDPEVLHSLQTQLKAQNSRIQQLFHKVAQQQRHLEKQHLRIQHLQSQFGLLDHKHLDHEVAKPARRKRLPEMAQPVDPAHNVSRLHRLPRDCQELFQVGERQSGLFEIQPQGSPPFLVNCKMTSDGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMAAEAAS

SEQ ID NO: 3 - DNA, 1201 bp, Homo sapiens
agtcccagctcagagccgcaacctgcacagccatgcccgggcaagaactcaggacgctgaatggctctcagatgctcctggtgttgctggtgctctcgtggctgccgcatgggggcgccctgtctctggccgaggcgagccgcgcaagtttcccgggaccctcagagttgcacaccgaagactccagattccgagagttgcggaaacgctacgaggacctgctaaccaggctgcgggccaaccagagctgggaagattcgaacaccgacctcgtcccggcccctgcagtccggatactcacgccagaagtgcggctgggatccggcggccacctgcacctgcgtatctctcgggccgcccttcccgaggggctccccgaggcctcccgccttcaccgggctctgttccggctgtccccgacggcgtcaaggtcgtgggacgtgacacgacctctgcggcgtcagctcagccttgcaagaccccaggcgcccgcgctgcacctgcgactgtcgccgccgccgtcgcagtcggaccaactgctggcagaatcttcgtccgcacggccccagctggagttgcacttgcggccgcaagccgccagggggcgccgcagagcgcgtgcgcgcaacggggaccactgtccgctcgggcccgggcgttgctgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcatatgagcagtcctggtccttccactgtgcacctgcgcgggggaggcgacctcagttgtcctgccctgtggaatgggctcaaggttcctgagacacccgattcctgcccaaacagctgtatttatataagtctgttatttattattaatttattggggtgaccttcttggggactcgggggctggtctgatggaactgtgtatttatttaaaactctggtgataaaaataaagctgtctgaactgttc

SEQ ID NO: 4 - protein, 308 aa, Homo sapiens [sequence omitted]

SEQ ID NO: 5 - DNA, 2580 bp, Homo sapiens
ggtatggcccggccggagttaaggccggggggaggcggcgagtcccgcggcggcggcgacgatggggctgcgtgcaggaggaacgctgggcagggccggcgcgggtcggggggcgcccgaggggcccgggccgagcggcggcgcgcagggcggcagcatccactcgggccgcatcgccgcggtgcacaacgtgccgctgagcgtgctcatccggccgctgccgtccgtgttggaccccgccaaggtgcagagcctcgtggacacgatccgggaggacccagacagcgtgccccccatcgatgtcctctggatcaaaggggcccagggaggtgactacttctactcctttgggggctgccaccgctacgcggcctaccagcaactgcagcgagagaccatccccgccaagcttgtccagtccactctctcagacctaagggtgtacctgggagcatccacaccagacttgcagtagcagcctccttggcacctgctgccaccttcaagagcccagaagacacacctggcctccagcaggctgggccatgcagaagggatagcaggggtgcattctctttgcacctggcgagagggtctgactctgggcacccctctcaccagctacaaggccttggactcactgtacagtgtgggagccccagttcccacctctgtgacaataggatcatggccttacccttgaagcattaccgagaaggagaacagagatgggcttgaagagccacgtgctgccggctccaaattcccaaggacaaggatccctctgcatttttgtctatgtaacctcttatatggactacattcagctgcaaggaaaggaaaaccttgattgcagtggtttaaacaaacagaagattgtttttccacatagcatggattctggagatgggtggctaatggtattggttcaacaactccacgaaggtaggggtcacgtcttggatccttttgccttaatctcagtgctcgttacttcatggtcccaagatggctgctgtatccccaagaatcatgtctgcgttcaaggaaggaggggtggaggaagaggaagggccaaactagctggacccgtcaccttctatcagaaagtaaaacctcgtcagaagtctgtttcctgctctctccctctgcatatcttcacttagatgcccttggcccgagccagctaccattgcacctctagctgcaaacaaagctaagacagcagggaacagaattgtcatggctgaatagaccaatcgtgttccatctactgagactggcacactgcctcctgcaataaaactgggatcccattaccaagagagaaatgcagaattgtgtaccagttagcttttgctgtgtaacaaaccatccccaaacttggcagctagaaacaaaccctgtattttcccacaatcctatgggttggcaatttgggctgggctcaacagggcagttctgctgctcacacctgggatccctcatggagctaaggtcagctgttacctcagctgggcctggatggtctaggatagccttactcacttgcctggcaggtgacaggctgttggctggaattgcttggttctcctccatgtggcctctccagcaggctagctcaggcttattcacatgatggcttcaggattccaaagagagtgagagtagaagctgaaagacttcttgagttcttggcctggaactgggactaggacagtgtcacttctgctaagttcttttggtcagagcaaatcacaaggctttacccagattcaagggatgagaaacagactacatgtcttgatgaggggaaccacaaagagcttgtggccatttttcacctatcacaaataattttggatgggtatttatttggataaaggtatttccctcttccccctttctctctgtctcatggggcctcactctgccaagttggaaggcactaagacattgtcctggccctcagggtctaggggaagaggtgttggggcaggaagtgagtctctccatgggctggacccactgtagtaggagtgcctccttgtctgcactgctggtatggggttaggccaggtaggacattccagaggggcttctgaaaaccaagagtccctggggaaagggaacagagtaaggcaggccttgttctcactgccctctaagggaacttggtcactcggcacttttaagcctcagtttctccagttcaataataaggacaagagcttttcccatgcattctctttccccgggaaagttgactgaggtgaccagtaatagaattgaaaagggagagtgtcttcagtgcaatgtggcatcctggattgggtcttggaacaaaaacaggacattagtgggaaaattggaaatctgaaaaaagtctgaattttagttaatataccaatttcagtctcttggttttgacagatgtaccatggtgatgtaagatgttgaccttggggtaggctgggtgaagggtatacaggaactctttgtactatctctgcaacttctctgtaaatctagtatcattccaaaataaaagtttatttaatttaaaaaaaaaaaaaaaaa

SEQ ID NO: 6 - protein, 137 aa, Homo sapiens
MGLRAGGTLGRAGAGRGAPEGPGPSGGAQGGSIHSGRIAAVHNVPLSVLIRPLPSVLDPAKVQSLVDTIREDPDSVPPIDVLWIKGAQGGDYFYSFGGCHRYAAYQQLQRETIPAKLVQSTLSDLRVYLGASTPDLQ

SEQ ID NO: 7 - DNA, 19 bp, Homo sapiens
cctgcagccattccaacct

SEQ ID NO: 8 - DNA, 21 bp, Homo sapiens
tcccttcttaagcttctgccg

SEQ ID NO: 9 - DNA, 26 bp, Homo sapiens
cagtacttccgctccatcccacagca

SEQ ID NO: 10 - DNA, 21 bp, Homo sapiens
agcagtcctggtccttccact

SEQ ID NO: 11 - DNA, 21 bp, Homo sapiens
aatcgggtgtctcaggaacct

SEQ ID NO: 12 - DNA, 27 bp, Homo sapiens
acctcagttgtcctgccctgtggaatg

SEQ ID NO: 13 - DNA, 21 bp, Homo sapiens
tgctcgttacttcatggtccc

SEQ ID NO: 14 - DNA, 19 bp, Homo sapiens
tccacccctccttccttga

SEQ ID NO: 15 - DNA, 28 bp, Homo sapiens
tggctgctgtatccccaagaatcatgtc

SEQ ID NO: 16 - DNA, 19 bp, Homo sapiens
agccccctttctgagtgca

SEQ ID NO: 17 - DNA, 19 bp, Homo sapiens
cccttggtccacgcctcta

SEQ ID NO: 18 - DNA, 27 bp, Homo sapiens
tgagatcgaggctgcaggatatgctca

SEQ ID NO: 19 - DNA, 2538 bp, Homo sapiens
ggcggaggttgcagtgagccaagatcataccactgcactccagcctgggcaacagagcgagagactgtctcggaagaaaaaaaaaaaaaaaaaggctgggggcagtggctcatgcctgtaatccccacactttagaggcagaggccgacggatgacttgaggtcaagagttcgagacctgcctggccaacatggtgaatcaccgtctctactaaaaacacaaaaattagccaggtgtggtggcgcatgactgtaatcccagctactcagtaggctgaggcaggaggcggaggttgcagtgagccgagattgtgccactgcactccaacctgggggacagagagaaactccgtctcaaaaaaaaaaaaaaaaagagatttaatcctagggccacccaatgaggagataggagggaacctcaaatccatttccaggaggaggagtttggggccatataaatgtatatatacaaatatatatatttttttaagatggagtcagccggccgtggtggctcacgcctctaatcccagtactttgggaggccgaggccagcggatcacaaggtcaggagattgagaccatcctagctaacacggtgaaaccccatctctactaaaaatacaaaaaaaaattagccaggcgtggtggggggtgctcatagtcccagctactcaggaggctgaggcaggagaatagcatgaacctgggagctggagcttgcaggttggagtgcaatggcatgatctcagctcactgcaacctccgcctcctgggttcaagcgattctcctgcctcaggctcccgagcagctgggattacaggcatgtgccaccacgaccggttaatttctgaaattttttagtagagacagggtttcatcatgttggtcaggctggtctcgaactcctgacctcaggtgatccgcccgccttggcctcccaaagtgctgggattacaggcgtgagccactgcacctggccaggggctagagttttaatttttaaggattttggagtgggctgaagtgtggaggtcattgcttggtgaaagagtgcaggaggtgaagtcacagacagtaagaaactgtattctcatgcagattccgttcctctgtggggtcttcacactggtgggtgtcattggttaaggatttcaaaaacatcttaagaaattcttctaaaaagtcttatgattctaaggtcggaaatcacatctatagcaaatggtcggtatcaggtgctacaagcaacttgcggtcacaaggaagtgggtcaaagtgcagcctgattagtgcttaattataactaagtttctgtccagaattctttttttttgagacagagttttgctcttgttgatcaggcggaagtgcaatggtgaaaacttggctcactgcaacctccgccctctgggttcaagcgattctcttgcttcagcctctcgagtagctgggattacaggcatgtaatcccaccaccaagcccagctaattttgtatatttagtagagacagggtttctccatgttggtcaggctagtctagaactcttgacgtcagatgatccacgtgcctcggcctcccaaagtgctgggattacaggcgagagccaccgtgcccggcgcagaattcttttttttagagatgaggtattgccatcttgcccagacttgtctcgaactcctgggctcaaacaatccacccacctcggcctcccaaagtgctgagattactgacataagccaccatgcctggcccccagaattatgaatcctgtgaggatggcttcaaggtgagcgctgagccagacaaaaggatggggtttgggagcaccctgcttagactggaaagataatgttggagaagacttcctggaagaggggctttttgcgtagagttttgaagaatgagtaggagttctccagaggaggatgagtaactgcaataacacccagtttatcaagtgcctcctatgtgtctggccctgtgctttacccctcatttgaccacctctccagtgagagtctcagtcctttttttcctggtgaggaaacaggcatggcagagaggcatgacacatcaaggttgcccttcctggctccatctagcccgttctcctctgcttcctttgtttttcaccatctttagcctttgaccccaaccaaaaagagaagagaggaaatcccatgggcatagacagccacctcttaaactcttgtctggaatttttcacatagtaacaatgtctttttttcctccaaaaagactcccaggctggaatggtgtcctcatatcgaggaagaggatactgaggcccagaaatgtgccctagctttactaggagcgcccccacctaaagatcctccccctaaatacacccccagaccccgcccagctgtggtcattggagtgtttactctgcaggcagggggaggagggcgggactgagcaggcggagacggacaaagtccggggactataaaggccggtccggcagcatctggtcagtcccagctcagagccgcaacctgcacagcc

SEQ ID NO: 20 - DNA, 3000 bp, Homo sapiens
cactgttttaaggacctcacctaacaccatctctcttcactcttccaacacccctgtgaattaggtgacattatgtccgttttacagattaggaaacaggcacagacaggtgactggcccaaggtcacacatctaataaaatagtgcaattttaacccaggcaaggtacagaactataggggatgacccagtatcaagtttcccttttgttttagttttggttccctgatagcaagtctgagacttgagtgtaagtcgtttatgtgggaggtgatccctggaagcagaagtaaggggtgggaggaatgggacagagggtgtgttagtgaagtcagttgtagcaatagggctgagtacttctggtctctgagaaggttccagaaagatctgaatggtctagaacacctctctgaatcgtccacctgtgacagcacaagactggcatttactcaccggctcctgcccccattagtgaatggcctggcacctctggaatgggcagagctcccacagggatgccccatctatcaaatggggatggagataacagactcgtctcataaagctgttaggttccatgagttaatatatgtaaagcgcatcaaaccatgctgggcacatggagagcactcagtaatgtatgctatgtatgacctgagggaagagcgttccatgcacagggaacagcaaatgtaaagggtctgaggccaggacacattcgctgtggaagaggcacagcaaggaggccagtgtgactagagttgactccggggcttgaggagtggaaggtcagaaggtgtgagccactgcatccagccagggaaggtttcttcactcttaaaaagaggctcgaagggatgtcctaacttccacttgtggacattcgcatgtaatgatgtgatgttttgagctacagtagccatcttgggaccatgaggggcacaccgcaggataacagaacaccaaggatgatggcacaaaaggatagacagaaccagggtcttttttttctgagagagtctcactctctcacccaggctgtagtgcaacggcatgatctcagctcactgcaacctctgcctctcggtttcaagcaattctcctgtctcagcctctggagtaactgggactacaggcgcgcgccaccatgcctgcctaatttttgtacttttggtagagacagggtttcactatgttggctaggctggtctcgaactcctggcctcaagtgattcgcctgcctcagccttccaaagtgctgggattacaggtgtgagccaccgggcccagccagaactggagtctttggtcacattgttcaactgcctctttaccaaccttggacatacctacctccaaacattttattatgtgagaaaacaaaccttcttattgcttaagccattttgaattgggccatagatatcttaacacagagaatttgataccaggaaatgaggcctccctgggtacaccatgatgatagaatcaggccagagctccacttttccactctagaaactgttatcctgccattcttacttcctgtaatgaggaagatgttttagaagggataacttccctcttgatgaaattctctaattctgaataattctggggctgaggtgtgggtgtcagttctacttcttgaaataaagtatcatttaggtgcgcacgggaattatgtttcgaaagggaaaggctggagtcttggtgtcagtgttctaaggttttgtgagagactaagaacccaaaataaatgatcttcaacagcaaaatggatgttatgcaattcacagagcactatgagcactctctcaattcacacaactttgagtgatgggcagcatgacttcattttacagacggggttcagaaagtgtcagcaactggtggaaggcaaagtagaaactcctggctccctccttcgtctccagttttctccagctggcagggtaagtatgcatttctccatgttcgtgccattcaggttgagaaggaagacaagcaaaccagacatgcttgtcttcgaccgagtctgggatcgtccaccttcctgcttggagttggtggttaagtggccccacagtttggactcgtgtgacctgactttcatttcagctgaccttgaacagcgggtctataactgcacctacttccagtagtgttgtaatgacacaaacagacagtgtggaacttgaaggcagggcccacccattgtcagcgcgactcctgagggctccgtgtgacagtcactgttgctgaaccaaggcagccaacggtggggtagaaggcggactctggagccagaccgtctggatttgaacgcggattcggccacttactagttccgtgatcttgggtacatcacttaaacctgtgtctcagtctcctcttctgtaaaatgggaataacagtcataccttccttgcagggttgttttgaggttcagatacgttaatacacgtgcaaaatgttcggaacgactccagacacagccggcgccagactggaagtggaaccgctggtggggatcataacctcgctggctaggaaccccagcagcggggcagcagcgcaccgcgggctttgccgactcaccctggctgggcaggtccgccgcgctctcccccgcccccgcccccgcggcctaagtcggcccggccggtgcgtcacccgcgcccctcccgccccgcgacctgcaaactcaccctgagtcagcccgcccgggcgtttccactccccggacgccgcgcggcgcaggggaggcgagaggcgccccccggtggagagcctgagccccgcgcgagtctggcggcacctggcgagcggagccggagtcgggctggggaccgcggggtcgaggccggaccgcggcggggtcgggggagaaacgcgcgctgccctggcacgggcccccccccccggccgcgcggaatggtatggcccggccggagttaaggccggggggaggcggcgagtcccgcggcggcggcgacg

SEQ ID NO: 21 - DNA, 21 bp, Homo sapiens
aacgatcaagtgacattgcta

SEQ ID NO: 22 - DNA, 21 bp, Homo sapiens
aactggatgacagtgatatct

(19) United States
(12) Patent Application Publication
(10) Pub. No.: US 2005/0191668 A1
(43) Pub. Date: Sep. 1, 2005

(54) Methods of using peroxisome proliferator-activated receptor alpha target genes

(76) Inventors: Lubing Zhou, Raritan, NJ (US); Ellen T. Cryan, East Brunswick, NJ (US); Keith T. Demarest, Flemington, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/036,746

(22) Filed: Jan. 14, 2005

Related U.S. Application Data
(60) Provisional application No. 60/536,474, filed on Jan. 14, 2004.

(51) Int. Cl. C12Q 1/68

(52) U.S. Cl. 435/6

(57) ABSTRACT

This invention features the identification of novel target genes for peroxisome proliferator-activated receptors alpha (PPARα) in human, and their use in treating or monitoring the treatment of metabolic abnormalities, as well as in identifying compounds useful for treating metabolic abnormalities.

### FIELD OF THE INVENTION

The present invention claims priority from U.S. Provisional Application No. 60/536,474 filed Jan. 14, 2004 entitled “Methods of Using Peroxisome Proliferator Activated Receptor Alpha Target Genes”, the contents of which are hereby incorporated herein by reference in their entirety. The invention relates to methods and compositions for treating or monitoring the treatment of metabolic abnormalities. The invention also relates to methods and compositions useful to identify compounds to treat metabolic abnormalities. In particular, methods of the invention relate to the use of three PPARα target genes Pa9, Pa13, and Pa21.

### BACKGROUND OF THE INVENTION

Metabolic abnormalities, such as, for example, obesity, diabetes, hypertension, and hyperlipidemia, pose a major public health problem in most industrialized societies. These metabolic abnormalities predispose individuals to coronary artery disease, stroke, and other cardiovascular diseases. Emerging data suggest that peroxisome proliferator-activated receptors (PPARs) are key players in lipid and glucose metabolism and are implicated in metabolic disorders.

PPARs are ligand-activated transcription factors belonging to the nuclear receptor superfamily. Activated PPARs heterodimerize with another nuclear receptor, the retinoid X receptor, and alter the transcription of target genes. Activated PPARs bind to specific DNA sequences known as peroxisome proliferator response elements (PPREs) located in the promoters of target genes to increase the rate of transcription initiation. Most PPREs identified so far consist of a direct repeat of the canonical AGGTCA sequence spaced by 1 nucleotide (Schoonjans et al., 1996, Biochim Biophys Acta, 1302:93-109). Three PPAR subtypes have been characterized: PPARα, δ, and γ. PPARα expression is seen in higher levels among cells of in brown adipose tissue, liver, heart, kidney and muscle, where it regulates fatty acid catabolism. PPARδ is expressed ubiquitously and its function is now being elucidated. PPARγ is mainly present in adipose tissue, colon and macrophage and is involved in adipocyte differentiation, lipid storage, and glucose homeostasis (Lee et al., 2003, supra).

Ligands for PPARs include dietary fatty acids, and a number of drugs used in the treatment of hyperlipidemia or type 2 diabetes. For example, fibrates, a group of serum triglyceride lowering agents, are synthetic agonists for PPARα. Examples of fibrates include, but are not limited to, gemfibrozil, bezafibrate, fenofibrate, clofibrate, and the newly developed micronized fenofibrate. Other synthetic agonists for PPARα include, but are not limited to, GW2331, WY14643 and L165041 (Mukherjee et al., 2002, J Steroid Biochem Mol Biol. 81(3): 217-25). Thiazolidinediones (TZDs), a class of insulin-sensitizing drugs, are high affinity PPARγ agonists. Examples of TZDs include, but are not limited to, Avandia (rosiglitazone) and Resulin (troglitazone).

Action of some PPARα agonists, such as fibrates, appear to be species specific (Vosper et al., 2002, Pharmacology & Therapeutics, 95:47-62). In rodents, administration of fibrates induces genes that are critical for lipid homeostasis, such as genes of HD (3-enoyl CoA dehydrogenase), ACOX (Acyl CoA Oxidase) and ME (Malic Enzyme) (Castelein et al, 1994 J. Biolo. Chem. 269: 26754-758). This is consistent with studies on PPARα-null mice, indicating that PPARα plays a critical role in maintaining constitutive activity of β-oxidative pathways in liver, adipocytes and skeletal muscles, the major target tissues for glucose and lipid metabolism in rodents (Leone et al., 1999, Proc. Natl. Acsd. Sci. U.S.A. 96:7473-7478). In human and several other “insensitive” species such as guinea pigs and marmosets, administration of fibrates does not appear to induce many of the genes induced by fibrates in rodents (Stael, et al., 1997, Current Pharmceutical Design 3:1-14). Regulation of peroxisomal fatty acid oxidation by fibrates is not observed in human hepatocytes or in livers of several other “insensitive” species. But, these “insensitive” animals do display potent lipid-lowering responses to fibrates.

Identifying novel genes regulated by PPARα in humans is critical to understanding the mechanism of fibrate treatment in human patients. Novel PPARα target genes will be valuable for clinically monitoring the activity of fibrates and other PPARα agonists. In addition, novel PPARα target genes will facilitate the rapid identification and development of new compounds that are useful for treating metabolic abnormalities. Furthermore, novel PPARα target genes will facilitate the development of new methods of treatment for metabolic abnormalities.

### SUMMARY OF THE INVENTION

It has now been discovered that genes Pa9, Pa13, and Pa21 designated herein are target genes of peroxisome proliferator-activated receptor alpha (PPARα) in human.

In one general aspect, the invention therefore provides a method of determining a biological activity of PPARα in a'subject. Such a method comprises the step of determining the expression level of a gene selected from the group consisting of a human Pa9 gene, a Pa13 gene, and a Pa21 gene in the subject.

In another general aspect, the invention provides a method of evaluating the effectiveness of a treatment for a metabolic abnormality in a subject. Such a method comprises the steps of: a) determining the expression level of a gene selected from the group consisting of a human Pa9 gene, a Pa13 gene, and a Pa21 gene in the subject during or after the treatment; and b) comparing the expression level determined in step a) with the expression level of the gene in the subject prior to the treatment; wherein an increase in the expression level of the gene in the subject during or after the treatment indicates that the treatment for the metabolic abnormality in the subject is effective.

The invention also features a kit for evaluating the effectiveness of a treatment for a metabolic abnormality in a subject. The kit can include a nucleic acid probe that hybridizes under stringent hybridization condition to a nucleic acid molecule selected from the group consisting of SEQ ID NO: 1, SEQ ID NO: 3, and SEQ ID NO: 5, or a complement thereof. The kit can also include an antibody that binds specifically to a polypeptide molecule selected from the group consisting of SEQ ID NO: 2, SEQ. ID NO: 4, and SEQ ID NO: 6. The kit can further include instructions for determining whether a treatment for a metabolic abnormality in a subject is effective or not.

In another general aspect, the invention provides a method of identifying a compound useful for treating a metabolic abnormality in a subject. One example of such method comprises the steps of: a) contacting a PPARα-responsive system with a solution comprising a buffer and a test compound; b) measuring from the PPARα-responsive system the expression level of a gene controlled by a regulatory sequence of a human Pa9 gene, a Pa13 gene, or a Pa21 gene; and c) comparing the result of step (b) with that of a control wherein the PPARα-responsive system is contacted with only the buffer. Another example of such method comprises the steps of: a) contacting a polypeptide encoded by a human Pa9 gene, a Pa13 gene, or a Pa21 gene with a solution comprising a buffer and a candidate or test compound; b) measuring the effect of the test compound on the activity of the polypeptide; and c) comparing the result of step (b) with that of a control wherein the polypeptide is contacted with only the buffer.

Another general aspect of the invention relates to methods of treating metabolic abnormalities in a subject. Such methods comprise the step of administering to the subject a therapeutic effective amount of a composition that alters the gene expression pattern or biological activity of a human Pa9 gene, a Pa13 gene, or a Pa21 gene in cells of the subject.

In yet another general aspect, the invention provides a PPARα-responsive system comprising a functional PPARα protein and a nucleic acid molecule comprising the coding sequence of a reporter gene operably linked to the regulatory sequence of a human Pa9 gene, a Pa13 gene, or a Pa21 gene. Also included in the invention is an isolated nucleic acid molecule comprising the coding sequence of a reporter gene operably linked to the regulatory sequence of a human Pa9 gene, a Pa13 gene, or a Pa21 gene.

Other aspects, features and advantages of the invention will be apparent from the following disclosure, including the detailed description of the invention and its preferred embodiments and the appended claims.

### BRIEF DESCRIPTION OF THE DRAWINGS

FIG. 1 shows the DNA sequence alignment between the Pa 9 microarray annotation sequence (GenBank accession number W30988) and the human FDRG gene sequence (GenBank accession number AX278133).

FIG. 2 shows the DNA sequence alignment between the Pa 13 microarray annotation sequence (GenBank accession number N26311) and the human TGF-beta superfamily protein gene sequence (GenBank accession number AB000584)

FIG. 3 shows the DNA sequence alignment between the Pa 21 microarray annotation sequence (GenBank accession number H49601) and the human C20orf139 gene sequence (GenBank accession number NM_{—}080725).

FIG. 4 illustrates that PPARα specific siRNA reduced the expression level of the PPARα gene in SKMU cells.

FIG. 5 illustrates that WY14643 had decreased induction of Pa9 gene expression in SKMU cells with reduced PPARα expression.

FIG. 6 illustrates that WY14643 had no detectable induction of Pa13 gene expression in SKMU cells with reduced PPARα expression.

FIG. 7 illustrates that WY14643 had no detectable induction of Pa21 gene expression in SKMU cells with reduced PPAR(X expression.

### DETAILED DESCRIPTION OF THE INVENTION

All publications cited herein are hereby incorporated by reference. Unless defined otherwise, all technical and scientific terms used herein have the same meaning as commonly understood to one of ordinary skill in the art to which this invention pertains.

As used herein, the terms “comprising”, “containing”, “having” and “including” are used in their open, non-limiting sense.

The following are abbreviations that are at times used in this specification:

bp=base pair

cDNA=complementary DNA

C20orf139=chromosome 20 open reading frame 139

ELISA=enzyme-linked immunoabsorbent assay

FDRG=Fibrinogen Domain Related protein

kb=kilobase; 1000 base pairs

nt=nucleotide

PAGE=polyacrylamide gel electrophoresis

PBMC=peripheral blood mononuclear cell

PCR=polymerase chain reaction

PPARα=peroxisome proliferator-activated receptor alpha

PPRE=peroxisome proliferator response element

RT PCR=Reverse transcription polymerase chain reaction

SDS=sodium dodecyl sulfate

SiRNA=small interfering RNA

SKMU=skeletal muscle

SSC=sodium chloride/sodium citrate

TZDs=Thiazolidinediones

UTR=untranslated region

WAT=white adipose tissue

“An activity”, “a biological activity”, or “a functional activity” of a polypeptide or nucleic acid refers to an activity exerted by a polypeptide or nucleic acid molecule as determined in vivo, or in vitro, according to standard techniques. Such activities can be a direct activity, such as an association with or an enzymatic activity on a second protein, or an indirect activity, such as a cellular signaling activity mediated by interaction of the protein with a second protein.

A “biological sample” as used herein refers to a sample containing or consisting of cells or tissue matter, such as cells or biological fluids isolated from a subject. The “subject” can be a mammal, such as a rat, a mouse, a monkey, or a human, who has been the object of treatment, observation or experiment. Examples of biological samples include, for example, sputum, blood, blood cells (e.g., white blood cells), amniotic fluid, plasma, semen, bone marrow, tissue or fine-needle biopsy samples, urine, peritoneal fluid, pleural fluid, and cell cultures. Biological samples may also include sections of tissues such as frozen sections taken for histological purposes.

In preferred embodiments, the biological sample is a “clinical sample,” which is a sample derived from a human patient. A biological sample may also be referred to as a “patient sample.” A test biological sample is the biological sample that has been the object of analysis, monitoring, or observation. A control biological sample can be either a positive or a negative control for the test biological sample. Often, the control biological sample contains the same type of tissues, cells and biological fluids of interest as that of the test biological sample.

A “cell” refers to at least one cell or a plurality of cells as appropriate for the sensitivity of the detection method. Cells suitable for the present invention may be bacterial, but are preferably eukaryotic, and are most preferably mammalian.

A “clone” is a population of cells derived from a single cell or common ancestor by mitosis. A “cell line” is a clone of a primary cell that is capable of stable growth in vitro for multiple generations.

A “gene” is a segment of DNA involved in producing a peptide, polypeptide, or protein, including the coding region as well as non-coding regions preceding (“5′UTR”) and following (“3′UTR”) coding regions. A “gene” may also include intervening non-coding sequences (“introns”) between individual coding segments (“exons”). “Promoter” refers to a regulatory sequence of DNA that is involved in the binding of RNA polymerase to initiate transcription of a gene. Promoters are often upstream (“5′ to”) the transcription initiation site of the gene. A “regulatory sequence” refers to the portion of a gene that can control the expression of the gene. A “regulatory sequence” can include promoters, enhancers and other expression control elements such as polyadenylation signals, ribosome binding sites (for bacterial expression), and/or, an operator. A “coding region” refers to a portion of a gene that codes for amino acids and the start and stop signals for translation via triplet-base codens.

“Gene expression microarray analysis” refers to an assay wherein a “microarray” of probe oligonucleotides is contacted with a nucleic acid sample of interest, e.g., a target sample, such as poly A mRNA from a particular tissue type, or a reverse transcript thereof. See, e.g., Nees et al. (2001), Curr Cancer Drug Targets, 1(2):155-75. Contact is carried out under hybridization conditions and unbound nucleic acid is removed. The resultant pattern of hybridized nucleic acid provides information regarding the genetic profile of the sample tested. Gene expression analysis can measure expression of thousands of genes simultaneously, providing extensive information on gene interaction and function. Gene expression analysis may find use in various applications, e.g., identifying expression of genes, correlating gene expression to a particular phenotype, screening for disease predisposition, and identifying the effect of a particular agent on cellular gene expression, such as in toxicity testing. “Microarray” as used herein refers to a substrate, e.g., a substantially planar substrate such as a biochip or gene chip, having a plurality of polymeric molecules spatially distributed over, and stably associated with or immobilized on, the surface of the substrate. Exemplary microarray formats include oligonucleotide arrays, and spotted arrays. Methods on gene expression microarray analysis are known to those skilled in the art. See, e.g., review by Yang et al. (2002), Nat Rev Genet 3(8): 579-88), or U.S. Pat. No. 6,004,755, which discloses methods for quantitative gene expression analysis using a DNA microarray.

A “human Pa9 gene” refers to a gene that (1) specifically hybridizes under stringent hybridization conditions to a nucleic acid molecule having a nucleotide sequence of SEQ ID NO: 1; (2) encodes a protein having an amino acid sequence of SEQ ID NO: 2; or (3) encodes a protein capable of binding to antibodies, e.g., polyclonal or monoclonal antibodies, raised against a protein having an amino acid sequence of SEQ ID NO: 2. “Stringent hybridization conditions” has the meaning known in the art, as described in Sambrook et al., Molecular Cloning: A Laboratory Manual, Second Edition, Cold Spring Harbor Laboratory, Cold Spring Harbor, N.Y., (1989). Exemplary stringent hybridization conditions include hybridization in 6× sodium chloride/sodium citrate (SSC) at about 45° C., followed by one or more washes in 0.2×SSC and 0.1% SDS at 50-65° C. The “human Pa9 gene” includes the human NL2 TIE ligand homologue polypeptide gene described in U.S. Pat. No. 6,348,350, and the human FDRG gene described in WO0177151. Exemplary human Pa9 genes include genes for structural and functional polymorphisms of human Pa9. “Polymorphism” refers to a set of genetic variants at a particular genetic locus among individuals in a population.

An “isolated” nucleic acid molecule is one that is substantially separated from other nucleic acid molecules. An “isolated” nucleic acid molecule can be, for example, a nucleic acid molecule that is free of at least one of the nucleotide sequences that naturally flank the nucleic acid molecule at its 5′ and 3′ ends in the genomic DNA of the organism from which the nucleic acid is derived. An isolated nucleic acid molecule includes, without limitation, a separate nucleic acid molecule (e.g., a cDNA or genomic DNA fragment produced by PCR or restriction endonuclease treatment) independent of other sequences, as well as a nucleic acid molecule that is incorporated into a vector, an autonomously replicating plasmid, a virus (e.g., a retrovirus, adenovirus, or herpes virus), or previously isolated nucleic acid incorporated into genomic DNA of a prokaryote or eukaryote. In addition, an isolated nucleic acid molecule can include a nucleic acid molecule that is part of a hybrid or fusion nucleic acid molecule.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced in one embodiment, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest. Isolated biologically active polypeptide can have several different physical forms. The isolated polypeptide can exist as a full-length nascent or unprocessed polypeptide, or as partially processed polypeptides or combinations of processed polypeptides. The full-length nascent polypeptide can be postranslationally modified by specific proteolytic cleavage events that results in the formation of fragments of the full-length nascent polypeptide. A fragment, or physical association of fragments can have the biological activity associated with the full-length polypeptide, however, the degree of biological activity associated with individual fragments can vary.

The term “labeled”, with regard to a labeled agent such as a nucleic acid probe or an antibody, is intended to encompass direct labeling of the agent by coupling (i.e., physically linking) a detectable substance to the agent as well as indirect labeling of the agent with another reagent that is directly labeled. Labels that are directly detectable include fluorescent labels and radioactive isotopes. Illustrative radioactive isotope labels include, e.g., ³⁵S, ³²P, and ³H. Preferred fluorescers are those absorbing light in wavelengths of from about 300 to 900 nm, more preferably from about 400 to 800 nm, and where the absorbance maximum is preferably at a wavelength ranging from about 500 to 800 nm. Exemplary fluorescers that may be used in singly labeled primers include fluorescein, rhodamine, BODIPY, cyanine dyes and the like. Fluorescers are further described in Smith et al., Nature (1986), 321: 647-679. Examples of indirect labeling include detection of a primary antibody using a fluorescently labeled secondary antibody, and end-labeling of a DNA probe with biotin such that it can be detected with fluorescently labeled streptavidin and the like. “End-labeled” with regard to a labeled nucleic acid molecule means that the label moiety is present at a region at least proximal to the terminus. Preferred end labels have the moiety at the 5′ terminus of the nucleic acid molecule. The labeling can also be at the 3′ terminus, using for example the enzyme terminal deoxynucleotidyl transferase.

A “Pa13 gene” refers to a gene that (1) specifically hybridizes under stringent hybridization conditions to a nucleic acid molecule having greater than about 60% nucleotide sequence identity to a human Pa13 cDNA (SEQ ID NO: 3); (2) encodes a protein having greater than about 60% amino acid sequence identity to a human Pa13 protein (SEQ ID NO: 4); or (3) encodes a protein capable of binding to antibodies, e.g., polyclonal or monoclonal antibodies, raised against the human Pa13 protein described herein. The human “Pa13 gene” includes the human TGF-beta superfamily protein (Yokoyama-Kobayashi et al., 1997,J Biochem (Tokyo). 122(3): 622-6, GenBank Protein_Id No: BAA19151).

The “Pa13 gene” can specifically hybridize under stringent hybridization conditions to a nucleic acid molecule having greater than about 65, 70, 75, 80, 85, 90, or 95 percent nucleotide sequence identity to SEQ ID NO: 3. In other embodiments, the Pa13 gene encodes a protein having greater than about 65, 70, 75, 80, 85, 90, or 95 percent amino acid sequence identity to SEQ ID NO: 4. Exemplary “Pa13 gene” includes genes for structural and functional polymorphisms of human Pa13, and its orthologs in other animals including rat, mouse, pig, dog and monkey.

A “Pa21 gene” refers to a gene that (1) specifically hybridizes under stringent hybridization conditions to a nucleic acid molecule having greater than about 60% nucleotide sequence identity to a human Pa21 cDNA (SEQ ID NO: 5); (2) encodes a protein having greater than about 60% amino acid sequence identity to a human Pa21 protein (SEQ ID NO: 6); or (3) encodes a protein capable of binding to antibodies, e.g., polyclonal or monoclonal antibodies, raised against the human Pa21 protein described herein. The human “Pa21 gene” includes the human C20orf 139 gene (Strausberg et al., 2002 Dec. 24, Proc Natl Acad Sci USA.; 99(26):16899-903. Epub 2002 Dec. 11., GenBank Protein_Id No: NP_{—}542763).

The “Pa21 gene” can specifically hybridize under stringent hybridization conditions to a nucleic acid molecule having greater than about 65, 70, 75, 80, 85, 90, or 95 percent nucleotide sequence identity to SEQ ID NO: 5. In other embodiments, the “Pa21 gene” encodes a protein having greater than about 65, 70, 75, 80, 85, 90, or 95 percent amino acid sequence identity to SEQ ID NO: 6. Exemplary “Pa21 gene” includes genes for structural and functional polymorphisms of human Pa21, and its orthologs in other animals including rat, mouse, pig, dog and monkey.

As used herein, a “peroxisome proliferator-activated receptor alpha” or “PPARα” refers to a polypeptide that: (1) has greater than about 60% amino acid sequence identity, to a human PPARα protein (Sher et al., 1993. Biochemistry. 32(21): 5598-604, GenBank Protein_ID No: NP_{—}005027); (2) is capable of binding to antibodies, e.g., polyclonal or monoclonal antibodies, raised against the human PPARα protein described herein; or (3) is encoded by a polynucleotide that specifically hybridizes under stringent hybridization conditions to a nucleic acid molecule having a sequence that has greater than about 60% nucleotide sequence identity to the human PPARα cDNA (GenBank nucleotide Accession No: NM_{—}005036).

The “PPARα” can be a polypeptide having greater than about 65, 70, 75, 80, 85, 90, or 95 percent amino acid sequence identity to human PPARα protein. In other embodiments, the “PPARα” is a polypeptide encoded by a polynucleotide that specifically hybridizes under stringent hybridization conditions to a nucleic acid molecule having a sequence that has greater than about 65, 70, 75, 80, 85, 90, or 95 percent nucleotide sequence identity to human PPARα cDNA. Exemplary “PPARα” protein includes structural and functional polymorphisms of human PPARα protein, and PPARα orthologs in other animals including a rat, mouse, pig, dog and monkey. A “functional PPARα” refers to the full length or any fragment of a PPARα protein that maintains the biological activity of a PPARα, such as upon binding to dietary fatty acids, fibrates, or other PPARα agonists, activating transcription of a host of target genes, including Pa9, Pa13, and Pa21 as disclosed herein.

The term “PPARα target genes” or “target genes of PPARα” comprises genes of any kind and origin the expression of which is regulated by PPARα. Examples of such genes are fatty acid acyl-CoA oxidase, 3-enoyl Co-A dehydrogenase, and malic enzyme in rodent, as well as Pa9, Pa13, and Pa21 genes described herein. In particular, the term “PPARα target genes” comprises the promoter sequences thereof and more particularly PPARα binding sequences thereof, for example the peroxisome proliferator response elements (PPREs). The target genes may be present in any DNA conformation. They can be present in cells, or occur in isolated fashion. The target genes can also be present in connection with further sequences, particularly with those coding for a reporter protein.

A “recombinant host cell” is a cell that has been transformed or transfected by an exogenous DNA sequence. As used herein, a cell has been “transformed” by exogenous DNA when such exogenous DNA has been introduced into the cell using any of a variety of techniques known to transport DNA into a prokaryotic or eukaryotic cell. Exogenous DNA may or may not be integrated (covalently linked) into chromosomal DNA making up the genome of the cell. For example, the exogenous DNA can be maintained on an episomal element, such as a plasmid. Alternatively, with respect to a stably transformed or transfected cell, the exogenous DNA has become integrated into the chromosome so that it is inherited by daughter cells through chromosome replication. This stability is demonstrated by the ability of the stably transformed or transfected cell to establish cell lines or clones comprised of a population of daughter cells containing the exogenous DNA. Recombinant host cells may be prokaryotic or eukaryotic, including bacteria such as E. coli, fungal cells such as yeast, mammalian cells including cell lines of human, bovine, porcine, monkey and rodent origin, and insect cells such as Drosophila and silkworm derived cell lines. It is further understood that the term “recombinant host cell” refers not only to the particular subject cell, but also to the progeny or potential progeny of such a cell. Because certain modifications can occur in succeeding generations due to either mutation or environmental influences, such progeny may not, in fact, be identical to the parent cell, but are still included within the scope of the term as used herein.

“Sequence” means the linear order in which monomers occur in. a polymer, for example, the order of amino acids in a polypeptide or the order of nucleotides in a polynucleotide.

“Sequence identity or similarity”, as known in the art, is the relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences.

To determine the percent identity or similarity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same or similar amino acid residue or nucleotide as the corresponding position in the second sequence, then the molecules are identical or similar at that position. The percent identity or similarity between the two sequences is a function of the number of identical or similar positions shared by the sequences (i.e., % identity=# of identical positions/total # of positions (e.g., overlapping positions) ×100). In one embodiment, the two sequences are the same length.

Both identity and similarity can be readily calculated. In calculating percent identity, only exact matches are counted. Methods commonly employed to determine identity or similarity between sequences include, e.g., those disclosed in Carillo et al. (1988), SIAM J. Applied Math. 48, 1073. Preferred methods to determine identity are designed to give the largest match between the sequences tested. Methods to determine identity and similarity are codified in computer programs.

An example of a mathematical algorithm utilized for the comparison of two sequences is the algorithm of Karlin et al. (1990), Proc. Natl. Acad. Sci. USA 87:2264-2268, modified as in Karlin et al. (1993), Proc. Natl. Acad. Sci. USA 90:5873-5877. Such an algorithm is incorporated into the NBLAST and XBLAST programs of Altschul et al. (1990), J Mol. Biol 215:403-410. To obtain gapped alignments for comparison purposes, Gapped BLAST can be utilized as described in Altschul et al. (1997), Nucleic Acids Res. 25:3389-3402. Alternatively, PSI-Blast can be used to perform an iterated search, which detects distant relationships between molecules. When utilizing BLAST, Gapped BLAST, and PSI-Blast programs, the default parameters of the respective programs (e.g., XBLAST and NBLAST) can be used. See, e.g., http://www.ncbi.nlm.nih.gov. Additionally, there is the FASTA method (Atschul et al. (1990), J. Molec. Biol. 215, 403) that can also be used.

Another example of a mathematical algorithm useful for the comparison of sequences is the algorithm of Myers et al. (1988), CABIOS 4:11-17. Such an algorithm is incorporated into the ALIGN program (version 2.0), which is part of the GCG sequence alignment software package (Devereux et al. (1984), Nucleic Acids Research 12(1), 387).

The term “substantially similar” as used herein refers to a polynucleotide or polypeptide sequence that includes the identical sequence as well as deletions, substitutions or additions thereto that result in a modified sequence that maintains any biologically active portion and possesses any of the conserved motifs thereof.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. Methods are known in the art for determining therapeutically effective doses for the instant pharmaceutical composition.

The term “vector” refers to a nucleic acid molecule capable of transporting another nucleic acid to which it has been linked. One type of vector is a “plasmid”, which refers to a circular double-stranded DNA loop into which additional DNA segments can be inserted. Another type of vector is a viral vector wherein additional DNA segments can be inserted. Certain vectors are capable of autonomous replication in a host cell into which they are introduced (e. g., bacterial vectors having a bacterial origin of replication and episomal mammalian vectors). Other vectors (e.g., non-episomal mammalian vectors) are integrated into the genome of a host cell upon introduction into the host cell, and are replicated along with the host genome. Moreover, certain vectors, such as expression vectors, are capable of directing the expression of genes to which they are operably linked. In general, vectors of utility in recombinant DNA techniques are often in the form of plasmids. However, the invention is intended to include other forms of vectors, such as viral vectors (e.g., replication defective retroviruses, adenoviruses and adeno-associated viruses), which serve equivalent functions.

Methods of Evaluating the Effectiveness of a Treatment for Metabolic Abnormalities

In one general aspect, the invention provides a method of determining biological activity of PPARα in a subject. Such a method comprises the step of determining the expression level of a gene selected from the group consisting of a human Pa9 gene, a Pa13 gene, and a Pa21 gene in the subject.

The invention provides a method to evaluate in a subject, the effectiveness of a treatment of a metabolic abnormality. The metabolic abnormality can be dyslipidaernia or a condition associated with dyslipidaemia, such as atherosclerosis, obesity, thrombosis or coronary artery disease, hypertension, angina, chronic renal failure, peripheral vascular disease, stroke, type II diabetes, impaired glucose tolerance (IGT), and metabolic syndrome (syndrome X).

In a preferred embodiment, the method of the invention can be used to evaluate the effectiveness of a treatment involving a PPARα agonist or a modulator. Examples of PPARα agonists include, but are not limited to, gemfibrozil, bezafibrate, fenofibrate, clofibrate, micronized fenofibrate, GW2331, WY14643, L165041, and the derivatives thereof.

A biological sample taken from a subject can be used to determine the expression level of a PPARα target gene selected from Pa9, Pa13, or Pa21 in the subject. Any suitable methods known to a skilled artisan can be used to obtain the biological sample, for example, via needle biopsy. For example, the biological sample can be obtained from brown adipose tissue, liver, heart, kidney or muscle, where PPARα expression is concentrated. The biological sample can also be obtained from adipose tissue or peripheral blood mononuclear cells (PBMC).

In some embodiments, the expression level of a PPARα target gene can be determined by measuring the mRNA amount of the gene. The amount of mRNA of a particular gene in a biological sample can be measured using a number of techniques. For example, mRNA can be measured by contacting the biological sample with a compound or an agent capable of specifically detecting the mRNA. Often a labeled nucleic acid probe capable of hybridizing specifically to the mRNA is used. For example, the nucleic acid probe specific for human Pa9 mRNA can be a full-length human Pa9 cDNA having nucleotide sequence of SEQ ID NO: 1, or a portion thereof, such as an oligonucleotide of at least 15, 30, 50, 100, 250 or 500 nucleotides in length that can hybridize to human Pa9 mRNA under stringent hybridization conditions. Under stringent conditions, the nucleic acid probe specific for human Pa9 mRNA will only hybridize to this mRNA but not the other mRNA species present in the biological sample. Useful nucleic acid probes for the invention include those capable of hybridizing to SEQ ID NO: 1, 3, or 5 under stringent hybridization conditions.

Another technique for determining the amount of mRNA from a particular gene in a biological sample is quantitative real-time reverse transcription polymerase chain reaction (RT-PCR). Complementary DNA (cDNA) from a gene, for example a human Pa 9 gene, can be prepared from the sample via reverse transcription. The cDNA can be amplified via PCR using oligonucleotide primers capable of hybridizing to the Pa9 cDNA under stringent hybridization conditions. Kits are commercially available that facilitate the RT-PCR, for example, the “One-Step RT-PCR Master Mix Reagent” kit from Applied Biosystems (Foster City, Calif.).

Over the decades, in situ hybridization has been used extensively to study the distribution and expression of mRNA within specific compartments of a cell or tissue. Types of nucleic acid probes used for in situ hybridization include single-stranded oligonucleotides, single-stranded RNA probes (riboprobes), or double-stranded cDNA sequences, of various lengths. Probes can be designed specifically against any known expressed nucleic acid sequence. A number of different radioisotope and non-isotopic labels are commercially available that may be used in in-situ hybridization. For a review of in-situ hybridization methods, see McNicol et al. (1997), J. Pathol 182(3): 250-61. Other useful techniques for determining the amount of mRNA from a particular gene in a biological sample include DNA microarray analysis, dot-blotting, and Northern hybridizations.

In some embodiments, the expression level of a PPARα target gene in a biological sample can be determined by measuring the amount of polypeptide encoded by the gene. The amount of a protein in a biological sample can be measured by contacting the biological sample with a compound or an agent capable of detecting the protein specifically. For example, a preferred agent for detecting a human Pa9 protein is an antibody capable of binding specifically to a portion of the polypeptide. In one preferred method, an antibody specific for a human Pa9 protein coupled to a detectable label is used for the detection of the human Pa9 protein. Antibodies can be polyclonal or monoclonal. A whole antibody molecule or a fragment thereof (e.g., Fab or F(ab′)₂) can be used. Antibodies are available through specialist laboratories. For example, antibodies directed against synthetic peptide sequences specific to Pa9, Pa13, or Pa21 protein can be developed within a relatively short time scale, enabling a greater degree of flexibility for studying these targets of interest.

Techniques for detection of a polypeptide such as the Pa9, Pa13, or Pa21 protein include enzyme linked immunosorbent assays (ELISAs), Western blots, immunoprecipitations and immunofluorescence, and immunohistochemistry. Details for performing these methods can be found in, for example, Sambrook et al. Molecular Cloning: A Laboratory Manual, Second Edition, Cold Spring Harbor Laboratory, Cold Spring Harbor, N.Y., (1989)).

In addition, the expression level of a PPARα target gene in a living human organ can be determined by quantitative noninvasive means, such as positron emission tomography (PET) imaging (Sedvall et al., (1988) Psychopharmacol. Ser., 5:27-33). For example, trace amounts of the Pa9 protein binding radiotracers can be injected intravenously into a subject, and the distribution of radiolabeling in brown adipose tissue, liver, heart, kidney, muscle, or other organs of the subject can be imaged. Procedures for PET imaging as well as other quantitative noninvasive imaging means are known to those skilled in the art (see review by Passchier et al., (2002) Methods 27:278).

Complete assay kits can be made available in which all reagents necessary for the detection of the expression level of a PPARα target gene are included, usually with an optimized protocol.

Kits for Determining the Effectiveness of a Treatment of a Metabolic Abnormality in a Subject

Thus, the invention also features a kit for determining the effectiveness of a treatment of a metabolic abnormality in a subject. Such a kit preferably comprises a compartmentalized carrier suitable to hold in close confinement at least one container. The carrier further comprises reagents capable of detecting the polypeptide or mRNA of a human Pa9 gene, a Pa13 gene, or a Pa21 gene in a biological sample and means for determining the amount of the polypeptide or mRNA in the sample (e.g., an enzyme or a substrate). The kit can also contain a control sample or a series of control samples that can be assayed and compared to the test sample contained. Each component of the kit can be enclosed within an individual container and all of the various containers are within a single package along with the instructions for determining whether a treatment for metabolic abnormalities in a subject is effective or not.

For an antibody-based kit, the kit can comprise, for example: (1) a first antibody (e.g., an antibody attached to a solid support) which binds to a polypeptide encoded by a gene of Pa9, Pa13, or Pa21; and, optionally; (2) a second, different antibody which binds to either the polypeptide or the first antibody and is conjugated to a detectable agent; and (3) a substantially purified polypeptide encoded by a gene of Pa9, Pa13, or Pa21 as positive control. For example, the antibody-based kit can comprise an antibody that binds specifically to a polypeptide of SEQ ID NO: 2, SEQ ID NO: 4, or SEQ ID NO: 6. The antibody can be polyclonal or monoclonal. Any suitable methods known to a skilled artisan can be used to develop the antibody.

For an oligonucleotide-based kit, the kit can comprise, for example, an oligonucleotide, e.g., a labeled oligonucleotide- which hybridizes to the mRNA of a gene Pa9, Pa13, or Pa21 under stringent hybridization conditions. For example, the kit can comprise a labeled oligonucleotide which hybridizes to the sequence SEQ ID NO: 1, SEQ ID NO: 2, or SEQ ID NO: 3, or complements thereof under stringent hybridization conditions. Alternatively, the kit can comprise a pair of primers useful for reverse transcription and amplification of a nucleic acid molecule from the mRNA of a gene Pa9, Pa13, or Pa21. For example, the kit can comprise a pair of primers useful for amplifying a nucleic acid molecule from SEQ ID NO: 1, SEQ ID NO: 2, or SEQ ID NO: 3.

The identification of genes Pa9, Pa13, and Pa21 as the novel target genes for PPARα, also allows for the development of new screening methods or assays for identifying compounds for their potential efficacy in treating a metabolic disorder.

Methods of Identifying Compounds Useful for Treating Metabolic Abnormalities

A general aspect of the invention relates to methods of identifying a compound useful for treating a metabolic abnormality in a subject. Such methods involve the identification of compounds that alter either the gene expression level of a human Pa9 gene, a Pa13 gene, or a Pa21 gene, or the biological activity of a polypeptide encoded by a human Pa9 gene, a Pa13 gene, or a Pa21 gene.

The compound identification methods can be performed using conventional laboratory formats or in assays adapted for high throughput. The term “high throughput” refers to an assay design that allows easy screening of multiple samples simultaneously, and can include the capacity for robotic manipulation. Another desired feature of high throughput assays is an assay design that is optimized to reduce reagent usage, or minimize the number of manipulations in order to achieve the analysis desired. Examples of assay formats include 96-well or 384-well plates, levitating droplets, and “lab on a chip” microchannel chips used for liquid-handling experiments. As known by those in the art, as miniaturization of plastic molds and liquid-handling devices are advanced, or as improved assay devices are designed, greater numbers of samples will be able to be screened more efficiently using the inventive assay.

Candidate compounds for screening can be selected from numerous chemical classes, preferably from classes of organic compounds. Although candidate compounds can be macromolecules, preferably the candidate compounds are small-molecule organic compounds, i.e., those having a molecular weight of greater than 50 and less than 2500. Candidate compounds have one or more functional chemical groups necessary for structural interactions with polypeptides. Preferred candidate compounds have at least an amine, carbonyl, hydroxyl or carboxyl group, preferably at least two such functional groups, and more preferably at least three such functional groups. The candidate compounds can comprise cyclic carbon or heterocyclic structural moieties and/or aromatic or polyaromatic structural moieties substituted with one or more of the above-exemplified functional groups. Candidate compounds also can be biomolecules such as peptides, saccharides, fatty acids, sterols, isoprenoids, purines, pyrimidines, derivatives or structural analogs of the above, or combinations thereof and the like. Where the compound is a nucleic acid, the compound is preferably a DNA or RNA molecule, although modified nucleic acids having non-natural bonds or subunits are also contemplated.

Candidate compounds may be obtained from a variety of sources, including libraries of synthetic or natural compounds. For example, numerous means are available for random and directed synthesis of a variety of organic compounds and biomolecules, including expression of randomized oligonucleotides, synthetic organic combinatorial libraries, phage display libraries of random peptides, and the like. Candidate compounds can also be obtained using any of the numerous approaches in combinatorial library methods known in the art, including: biological libraries; spatially addressable parallel solid-phase or, solution-phase libraries: synthetic library methods requiring deconvolution; the “one-bead one-compound” library method; and synthetic library methods using affinity chromatography selection (see, e.g., Lam (1997), Anticancer Drug Des. 12:145). Alternatively, libraries of natural compounds in the form of bacterial, fungal, plant and animal extracts are available or may be routinely produced. Additionally, natural and synthetically produced libraries and compounds can be routinely modified through conventional chemical, physical, and biochemical means.

Further, known pharmacological agents can be subjected to directed or random chemical modifications, such as acylation, alkylation, esterification, amidification, etc., to produce structural analogs of the agents. Candidate compounds can be selected randomly or can be based on existing compounds that bind to and/or modulate the function of PPARα activity. For example, a source of candidate agents can be libraries of molecules based on known activators or inhibitors for PPARα, in which the structure of the compound is changed at one or more positions of the molecule to contain more or fewer chemical moieties or different chemical moieties. The structural changes made to the molecules in creating the libraries of analog activators/inhibitors can be directed, random, or a combination of both directed and random substitutions and/or additions.

A variety of other reagents also can be included in the mixture. These include reagents such as salts, buffers, neutral proteins (e.g., albumin), and detergents that can be used to facilitate optimal protein-protein and/or protein-nucleic acid binding. Such a reagent can also reduce non-specific or background interactions of the reaction components. Other reagents that improve the efficiency of the assay, such as nuclease inhibitors, antimicrobial agents, and the like, can also be used.

Examples of methods for the synthesis of molecular libraries can be found in the art, for example in: Zuckermann et al. (1994), J Med. Chem. 37:2678. Libraries of compounds can be presented in solution (e.g., Houghten (1992), Biotechniques 13:412-421), or on beads (Lam (1991), Nature 354:82-84), chips (Fodor (1993), Nature 364:555-556), bacteria (U.S. Pat. No. 5,223,409), spores (U.S. Pat. No. 5,571,698), plasmids (Cull et al. (1992), Proc. Natl. Acad. Sci. USA 89:1865-1869) or phage (see e.g., Scott and Smith (1990), Science 249:386-390).

In one embodiment, the invention provides a method of identifying a compound useful for treating a metabolic abnormality in a subject, comprising the steps of: a) contacting a PPARα-responsive system with a solution comprising a buffer and a test compound; b) measuring the expression level of a gene controlled by a regulatory sequence of a human Pa9 gene, a Pa13 gene, or a Pa21 gene from the PPARα-responsive system; and optionally the result of step (b) with that of a control wherein the PPARα-responsive system is measured in the absence of test compound.

The identified compound useful for treating a metabolic abnormality includes any compound that can increase or decrease gene expression of a human Pa9 gene, a Pa13 gene, or a Pa21 gene from a PPARα-responsive system. For example, the compound can bind to PPARα in a PPARα-responsive system and regulate a biological activity of PPARα directly, resulting in increased or decreased expression of a human Pa9 gene, a Pa13 gene, or a Pa21 gene from a PPARα-responsive system. The compound can also bind to a cellular component that interacts with PPARα in a PPARα-responsive system and regulate a biological activity associated with the PPARα indirectly, resulting in increased or decreased expression of a human Pa9 gene, a Pa13 gene, or a Pa21 gene from the PPARα-responsive system. The compound can further increase or decrease the expression of a human Pa9 gene, a Pa13 gene, or a Pa21 gene, from the PPARα-responsive system, via a mechanism independent of the biological activity of the PPARα.

A “PPARα-responsive system” as used in its broadest sense refers to single cells, tissues, and complex multicellular organisms such as mammals, that are responsive to the stimulation of a PPARα gene, such as for example when using a PPARα agonist. In a preferred embodiment, the PPARα-responsive system is an animal, a tissue, or a cell comprising a functional PPARα protein and at least one PPARα target gene selected from the group consisting of Pa9, Pa13, and Pa21 genes. The PPARα-responsive system can be a natural host for an endogenous PPARα and an endogenous PPARα target gene Pa9, Pa13, or Pa21. For example, human hepatocyte HuH7 cells, human primary adipocytes, and human primary skeletal muscle cells (SKMC) are all natural PPARα-responsive systems-that can be used in the invention. The PPARα-responsive system can also be a recombinant host cell for PPARα. Any suitable method known to a skilled artisan may be used to obtain such a PPARα responsive system with a recombinant PPARα. For example, the PPARα-responsive system can be constructed by introducing an exogenous DNA encoding a functional PPARα protein into a natural host cell for at least one endogenous PPARα target gene Pa9, Pa13, or Pa21. The expression level of the Pa9 gene, Pa13 gene, or Pa21 gene from such a PPARα-responsive system can be measured either by the amount of mRNA or protein of the gene from the PPARα-responsive system using methods described supra.

In another embodiment, the PPARα-responsive system comprises a functional PPARα protein and a nucleic acid molecule comprising the coding sequence of a reporter gene operably linked to the regulatory sequence of a human Pa9 gene, a Pa13 gene, or a Pa21 gene. Such a system allows for transcriptional regulation of a reporter gene in response to a PPARα modulator. Therefore, the expression level of a PPARα target gene can be measured indirectly via a reporter activity. For example, when a luciferase (luc) gene is used as the reporter gene, the expression level of a PPARα target gene can be measured as the amount of bioluminescence from the PPARα-responsive system. Other reporter genes include, but are not limited to, genes of green fluorescent protein (GFP), β-galactosidase (lacZ), chloramphenicol acetyltransferase (cat), β-glucuronidase, neomycin phosphotransferase, and guanine xanthine phosphoribosyl-transferase. The biological activity of the reporter can be easily measured. Kits are available commercially to facilitate the measurement of the reporter activity.

Any suitable methods known to a skilled artisan may be used to construct a nucleic acid comprising a coding sequence of a reporter gene operably linked to a regulatory sequence of a human Pa9 gene, a Pa13 gene, or a Pa21 gene. The regulatory sequence of a human Pa9 gene, a Pa13 gene, or a Pa21 gene includes any nucleotide sequence that is naturally associated with and controls the gene expression of the human Pa9 gene, the Pa13 gene, or the Pa21, respectively. Preferably, the regulatory sequence comprises one or more potential PPREs. For example, a nucleotide sequence of SEQ ID NO. 19 or a portion thereof represents a regulatory sequence for the human Pa13 gene; and a nucleotide sequence of SEQ ID NO: 20 or a portion thereof represents a regulatory sequence for the human Pa21 gene.

In another embodiment, the invention provides a method of identifying a compound useful for treating a metabolic abnormality in a subject, comprising the steps of: a) contacting a polypeptide encoded by a human Pa9, gene, a Pa13 gene, or a Pa21 gene with a solution comprising a buffer and a test compound; b) measuring the effect of the test compound on the activity of the polypeptide; and c) comparing the result of step (b) with that of a control lacking the test compound.

Binding assays can be used to identify a compound that binds to a polypeptide encoded by a human Pa9 gene, a Pa13 gene, or a Pa21 gene, and potentially is capable of increasing or decreasing the biological activity of the polypeptide. One exemplary binding assay comprises the steps of: (a) incubating a test compound with a polypeptide encoded by a human Pa9 gene, a Pa13 gene, or a Pa21 gene and a labeled ligand for the polypeptide; (b) separating the polypeptide from the unbound labeled ligand; and (c) identifying a compound that inhibits ligand binding to the polypeptide by a reduction in the amount of labeled ligand binding to the polypeptide. An example of the labeled ligand for a polypeptide is a labeled antibody specific for the polypeptide. A host cell (recombinant or native) that expresses a human Pa9 gene, a Pa13 gene, or a Pa21 gene can be used for the binding assay. Preferably, cell membranes prepared from the host cell can be used for the binding assay. More preferably, a substantially purified polypeptide encoded by a human Pa9 gene, a Pa13 gene, or a Pa21 gene can be used for the binding assay.

Separation of the polypeptide from unbound labeled ligand can be accomplished in a variety of ways. Conveniently, at least one of the components is immobilized on a solid substrate, from which the unbound components can be easily separated. The solid substrate can be made of a wide variety of materials and in a wide variety of shapes, e.g., microtiter plate, microbead, dipstick, resin particle, etc. The substrate preferably is chosen to maximize signal to noise ratios, primarily to minimize background binding, as well as for ease of separation and cost.

Separation can be effected for example, by removing a bead or dipstick from a reservoir, emptying or diluting a reservoir such as a microtiter plate well, or rinsing a bead, particle, chromatographic column or filter with a wash solution or solvent. The separation step preferably includes multiple rinses or washes. For example, when the solid substrate is a microtiter plate, the wells can be washed several times with a washing solution, that typically includes those components of the incubation mixture that do not participate in specific bindings such as salts, buffer, detergent, non-specific protein, etc. Where the solid substrate is a magnetic bead, the beads can be washed one or more times with a washing solution and isolated using a magnet.

A wide variety of labels can be used to label the ligand, such as those that provide direct detection (e.g., radioactivity, luminescence, optical or electron density, etc), or indirect detection (e.g., epitope tag such as the FLAG epitope, enzyme tag such as horseradish peroxidase, etc.).

In more than one embodiment of the above assay methods of the present invention, it can be desirable to immobilize either the polypeptide or its ligand to facilitate separation of complexed from uncomplexed forms of the polypeptide, as well as to accommodate automation of the assay. Binding of a test compound to a polypeptide, or interaction of a polypeptide with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein of the polypeptide can be provided which adds a domain that allows one or both of the polypeptide and its ligand to be bound to a matrix. For example, a fusion protein of the polypeptide with glutathione-S-transferase can be adsorbed onto glutathione sepharose beads (Sigma Chemical; St. Louis, Mo.) or glutathione derivatized microtitre plates, which are then combined with the test compound or the test compound and the labeled ligand for the polypeptide, and the mixture incubated under conditions conducive to complex formation (e.g., at physiological conditions for salt and pH). Following incubation, the beads or microtitre plate wells are washed to remove any unbound components and complex formation is measured either directly or indirectly, for example, as described above. Alternatively, the complexes can be dissociated from the matrix, and the level of binding or activity of the polypeptide of the invention can be determined using standard techniques.

Other techniques for immobilizing proteins on matrices can also be used in the screening assays of the invention. For example, either the polypeptide or its ligand can be immobilized utilizing biotin and streptavidin conjugation.

Biotinylated polypeptide or target molecules can be prepared from biotin-NHS (N-hydroxy-suceinimide) using techniques well known in the art (e.g., biotinylation kit, Pierce Chemicals; Rockford, Ill.), and immobilized in the wells of streptavidin-coated 96 well plates (Pierce Chemical). Alternatively, antibodies reactive with the polypeptide but which do not interfere with binding of the polypeptide to its target molecule can be derivatized to the wells of the plate and polypeptide can be trapped in the wells by antibody conjugation. Methods for detecting such complexes, in addition to those described above for the GST-immobilized complexes, include immunodetection of complexes using antibodies reactive with the polypeptide of the invention or target molecule, as well as enzyme-linked assays that rely on detecting an enzymatic activity associated with the polypeptide.

The identification of genes Pa9, Pa13, and Pa21 as the novel target genes for PPARα, also allows for the development of new methods for treating metabolic abnormalities.

Methods of Treating Metabolic Abnormalities

Thus, another general aspect of the invention relates to methods of treating metabolic abnormalities in a subject. Such methods comprise the step of administering to the subject a therapeutic effective amount of a composition that alters the gene expression pattern or biological activity of a human Pa9 gene, a Pa13 gene, or a Pa21 gene in cells of the subject.

In one embodiment, the methods of treatment comprises the step of administering to the subject a compound capable of increasing or decreasing the expression or biological activity of a human Pa9 gene, a Pa13 gene, or a Pa21 gene. Such compounds can be identified using the methods of compound identification described supra.

In another embodiment, gene therapy targeting a human Pa9 gene, a Pa13 gene, or a Pa21 gene in a cell can be used to treat a metabolic abnormality in a subject. For example, antisense therapy can be used to decrease the expression of a human Pa9 gene, a Pa13 gene, or a Pa21 gene in a cell, when decreased expression or activity of the human Pa9 gene, Pa13 gene, or Pa21 gene, is desirable.

The principle of antisense-based strategies is based on the hypothesis that sequence-specific suppression of gene expression can be achieved by intracellular hybridization between mRNA and a complementary antisense species. The formation of a hybrid RNA duplex can then interfere with the processing/transport/translation and/or stability of the target mRNA, such as that of the gene Pa9, Pa13, or Pa21. Hybridization is required for the antisense effect to occur. Antisense strategies can use a variety of approaches including the use of antisense oligonucleotides, injection of antisense RNA and transfection of antisense RNA expression vectors. Phenotypic effects induced by antisense hybridization to a sense strand are based on changes in criteria such as protein levels, protein activity measurement, and target mRNA levels.

An antisense nucleic acid can be complementary to an entire coding strand of a target gene, or to only a portion thereof. An antisense nucleic acid molecule can also be complementary to all or part of a non-coding region of the coding strand of a target gene. The non-coding regions (“5′ and 3′ UTRs”) are the 5′ and 3′ sequences which flank the coding region and are not translated into amino acids. Preferably, the non-coding region is a regulatory region for the transcription or translation of the target gene.

An antisense oligonucleotide can be, for example, about 15, 25, 35, 45 or 65 nucleotides or more in length taken from the complementary sequence of SEQ ID NO: 1, 3 or 5. It is preferred that the sequence be at least 18 nucleotides in length in order to achieve sufficiently strong annealing to the target mRNA sequence to prevent translation of the sequence. (Izant et al., 1984, Cell, 36:1007-1015; Rosenberg et al., 1985, Nature, 313:703-706). An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxytnethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylecytosine,. 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3 )w, and 2,6-diaminopurine. An antisense nucleic acid molecule can be a CC-anomeric nucleic acid molecule. A CC-anomeric nucleic acid molecule forms specific double- stranded hybrids with complementary RNA in which, contrary to the usual P-units, the strands run parallel to each other (Gaultier et al. (1987) Nucleic Acids Res. 15:6625-664 1). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

Alternatively, the antisense nucleic acid can also be produced biologically using an expression vector into which a nucleic acid has been subcloned in an antisense orientation as described supra. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. To achieve sufficient intracellular concentrations of the antisense molecules, vector constructs in which the antisense nucleic acid molecule is placed under the control of a strong pol II or pol III promoter are preferred. For a discussion of the regulation of gene expression using antisense genes see Weintraub et al. (1985, Trends in Genetics, Vol. 1(1), pp. 22-25).

Typically, antisense nucleic acid is administered to a subject by microinjection, liposome encapsulation or generated in situ by expression from vectors harboring the antisense sequence. An example of a route of administration of antisense nucleic acid molecules includes direct injection at a tissue site. The antisense nucleic acid can be ligated into viral vectors that mediate transfer of the antisense nucleic acid when the viral vectors are introduced into host cells. Suitable viral vectors include retrovirus, adenovirus, adeno-associated virus, herpes virus, vaccinia virus, polio virus and the like. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens.

Once inside the cell, antisense nucleic acid molecules hybridize with or bind to cellular mRNA and/or genomic DNA encoding a Pa9, Pa13, or Pa21 protein to thereby inhibit expression, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix.

In a preferred embodiment, the method involves the use of small interfering RNA (siRNA). Many organisms possess mechanisms to silence gene expression when double-stranded RNA (dsRNA) corresponding to the gene is present in the cell through a process known as RNA interference. The technique of using dsRNA to reduce the activity of a specific gene was first developed using the worm C. elegans and has been termed RNA interference, or RNAi (Fire, et al., (1998), Nature 391: 806-811). RNAi has since been found to be useful in many organisms, and recently has been extended to mammalian cells (see review by Moss, (2001), Curr Biol 11: R772-5). An important advance was made when RNAi was shown to involve the generation of small RNAs of 21-25 nucleotides (Hammond et al., (2000) Nature 404: 293-6; Zamore et al., (2000) Cell 101: 25-33). These small interfering RNAs, or siRNAs, may initially be derived from a larger dsRNA that begins the process, and are complementary to the target RNA that is eventually degraded. The siRNAs are themselves double-stranded with short overhangs at each end. They act as guide RNAs, directing a single cleavage of the target in the region of complementarity (Elbashir et al., (2001) Genes Dev 15: 188-200; Zamore et al., (2000) Cell 101: 25-33).

SiRNAs comprising about 21-25 nucleotides complementary to the nucleotide sequences of SEQ ID NO: 1, 3, or 5 can be used in the methods of treatment of this invention. Methods of producing siRNA are known to those skilled in the art. For example, WO0175164 A2 described methods of producing siRNA of 21-23 nucleotides (nt) in length from an in vitro system, and using such siRNA to interfere with mRNA of a gene in a cell or organism. The siRNA can also be made in vivo from a mammalian cell using a stable expression system. For example, a vector system, named pSUPER, that directs the synthesis of siRNAs in mammalian cells, was recently reported (Brummelkamp et al., (2002) Science 296: 550-3). An example of using siRNA to reduce gene expression in a cell is shown in Example 3.

The present invention provides a method of treating a metabolic abnormality in a subject, comprising the steps of (a) introducing siRNA that targets the mRNA of a human Pa9 gene, a Pa13 gene, or a Pa21 gene, for degradation into a cell of the subject; and (b) maintaining the cell produced in (a) under conditions under which siRNA interference of the mRNA of the human Pa9 gene, Pa13 gene, or Pa21 gene in the cell of the subject occurs. The siRNA can be introduced into the cell of the subject using procedures similarly to those for the anti-sense nucleic acids described herein.

In another embodiment, gene therapy can be used to increase the expression of a human Pa9 gene, a Pa13 gene, or a Pa21 gene by introducing a nucleic acid molecule capable of expressing the gene into a cell of a subject. Such gene therapy can be particularly useful for the treatment of diseases where it is beneficial to elevate the expression or biological activity of the human Pa9 gene, Pa13 gene, or Pa21 gene.

A procedure for performing ex vivo gene therapy is outlined in U.S. Pat. No. 5,399,346 and also in exhibits submitted in the file history of that patent, all of which are publicly available documents. In general, gene therapy can involve introduction in vitro of a functional copy of a gene into a cell(s) of a subject, and returning the genetically engineered cell(s) to the subject. The functional copy of the gene is under operable control of regulatory elements, which permit expression of the gene in the genetically engineered cell(s). Numerous transfection and transduction techniques as well as appropriate expression vectors are well known to those of ordinary skill in the art, some of which are described in PCT application WO95/00654. In vivo gene therapy uses vectors such as adenovirus, retroviruses, vaccinia virus, bovine papilloma virus, and herpes virus such as Epstein-Barr virus. Gene transfer can also be achieved using non-viral means requiring infection in vitro. Such means can include calcium phosphate, DEAE dextran, electroporation, and protoplast fusion. Targeted liposomes can also be potentially beneficial for delivery of DNA into a cell.

As one example, a DNA molecule encoding a target gene can be first cloned into a retroviral vector. The expression of the target gene from the vector can be driven from its endogenous promoter or from the retroviral long terminal repeat or from a promoter specific for certain target cells. The vector can then be introduced into a cell of a subject to successfully express the target gene in the target cells. The gene can be preferably delivered to those cells in a form which can be used by the cell to encode sufficient protein to provide effective function. Retroviral vectors are often a preferred gene delivery vector for gene therapy especially because of their high efficiency of infection and stable integration and expression. Alternatively, the DNA molecule encoding a target gene can be transferred into cells for gene therapy by non-viral techniques including receptor-mediated targeted DNA transfer using ligand-DNA conjugates or adenovirus-ligand-DNA conjugates, lipofection membrane fusion or direct microinjection. These procedures and variations thereof are suitable for ex vivo as well as in vivo gene therapy. Protocols for molecular methodology of gene therapy suitable for use with the methods of the invention are described in Gene Therapy Protocols, edited by Paul D. Robbins, Human press, Totowa N.J., 1996.

During treatment, the effective amount of nucleic acid molecules of the invention administered to individuals can vary according to a variety of factors including type, species, age, weight, sex and medical condition of the patient; the severity of the condition to be treated; the route of administration; the renal and hepatic function of the patient; and the particular nucleic acid molecule thereof employed. A physician or veterinarian of specialized skill in gene therapy can determine and prescribe the effective amount required to prevent, counter or arrest the progress of the condition. Optimal precision in achieving concentrations within the range that yields efficacy without toxicity requires a regimen based on the kinetics of the nucleic acid molecule's availability to target sites. This involves a consideration of the distribution, equilibrium, and elimination of the nucleic acid molecule involved in gene therapy.

The gene therapy disclosed herein can be used alone at appropriate dosages defined by routine testing in order to obtain optimal increase or decrease of the PPARα target gene activity while minimizing any potential toxicity. In addition, co-administration or sequential administration of other agents may be desirable. The dosages of administration are adjusted when several agents are combined to achieve desired effects. Dosages of these various agents can be independently optimized and combined to achieve a synergistic result wherein the pathology is reduced more than it would be if either agent were used alone.

The following examples illustrate the present invention without, however, limiting the same thereto.

### EXAMPLE 1

### Identification of Target Genes Regulated by PPARα Agonists in Human Cells

DNA microarray technique was used to identify the target genes regulated by fibrates or other PPARα agonists in human cells.

Human hepatocyte HuH7 cells, which are epithelial cells of liver, were obtained from Japan Health Science Research Resources Bank (Osaka, Japan). Human primary adipocytes, which are connective tissue cells specialized for the synthesis and storage of fat, were obtained from Zen-Bio, Inc (Research Triangle Park, NC). Human primary skeletal muscle cells (SKMC) were obtained from Cambrex Bio Science (Walkersville, Md.). PPARα agonists, Wy14643 (Agatha, et al, 2000, Archives of Biochemistry and Biophysics 380:353-359) and fenofibrates were obtained from Cayman Chemical (Ann Arbor, Mich.) and Sigma (St Louis, Mo.), respectively.

Human HuH7 cells were cultured in Dulbecco's Modified Eagle Medium with 10% Fetal Bovine Serum (Gibco, N.Y.). Cells were treated with charcoal-stripped, delipidated calf serum (5%) (Sigma, St Louis, Mo.) for about 4-18 hours. Then, cells were treated with different concentrations of PPARα agonist (Wy14643 or fenofibrates) for about 20-24 h. Human SKM cells were cultured in Skeletal Muscle Growth Medium (Cambrex Bio Science, Walkersville, Md.), and were treated with different concentrations of PPARα agonist (Wy14643 or fenofibrates) for about 18-20 hours. Human primary adipocytes were cultured in Preadipocyte Medium, (Cat #PM_{—}1, Zen-Bio, Inc., Research Triangle Park, NC), and were treated with different concentrations of PPARα agonist (Wy14643 or fenofibrates) for about 18-20 hours.

Total RNA was isolated from the cells and subjected to DNA microarray. JJPRD version MEGA and MEGAB DNA microarrays (about 6,000 genes/chip) were used. Total about 12,000 genes were analyzed. More than 40 pieces of Target chips and MEG-GA chips were used in the experiments analyses. The expression levels of over 12,000 genes were measured from cells treated with PPARα ligand and compared with those from cells treated with the vehicle control. A systematic search for genes whose expression levels were either increased or decreased by PPARα agonists was performed using the software OMNI-Viz (OmniViz, Inc. Richland, Wash.). About 24 genes were identified to have more than about 1.8-fold change in gene expression due to the PPARα agonist treatment, in one, two, or all three cells analyzed: human adipocyts, SKM and HuH7 cells. TaqMan probes were designed based on the cDNA sequences of those genes and Real Time quantitative PCR (RTQPCR) was used to confirm the gene expression alterations. Three genes, Pa9, Pa13, and Pa21, were confirmed to be regulated by PPARα agonists in all three human cells.

Total RNA was purified from cultured cells following the protocol provided by the manufacturer (Trizon method, Invitrogen Inc. Ca). The polymerase chain reaction (PCR) primers and fluorescent probes (TagMan probes) for Pa9, Pa13, and Pa21 were designed from GenBank sequences of accession numbers W30988, AB000584, and NM_{—}080725, respectively. The primers and probes were designed using the software PrimerDesign (Applied Biosystems, Foster City, Calif.). The PCR primers for Pa9 were SEQ ID NO: 7, (5-CCTGCAGCCA TTCCAACCT) and SEQ ID NO: 8 (5-TCCCTTCTTA AGCTTCTGCCG). The fluorescent probe for Pa9 was SEQ ID NO: 9 (6FAM-CAGTACTTCC GCTCCATCCC ACAGCA). The PCR primers for Pa13 were SEQ ID NO: 10 (5-AGCAGTCCTG GTCCTTCCAC, T) and SEQ ID NO: 11 (5-AATCGGGTGT CTCAGGAACC T). The fluorescent probe for Pa13 was SEQ ID NO: 12 (6FAM-ACCTCAGTTG TCCTGCCCTG TGGAATG). The PCR primers for Pa21 were SEQ ID NO: 13 (5-TGCTCGTTAC TTCATGGTCC C) and SEQ ID NO: 14 (5-TCCACCCCTC CTTCCTTGA). The fluorescent probe for Pa21 was SEQ ID NO: 15 (6FAM-TGGCTGCTGT ATCCCCAAGA ATCATGTC). All primers and probes were synthesized by Keystone Labs (Camarillo, Calif.).

Reverse transcription (RT) and PCR were conducted in one step using “One-Step RT-PCR Master Mix Reagent” kit (Applied Biosystems, Foster City, Calif.) in ABI PRISM 7700 Sequence Detection System (Applied Biosystems). Each reaction was performed in a total volume of 25 μl containing 40 ng of total RNA. The reaction condition was: 48° C. for 30 minutes, 60° C. for 30 minutes, 94° C. for 5 minutes, followed by 40 cycles of 94° C. for 20 seconds and 60° C. for 1 minute. The primers for 18S ribosomal RNA were developed and provided by Applied Biosystems. The measurement of human 18S ribosomal RNA was used as an internal control for normalization of measuring and loading errors. PCR data were collected by Ct value (the cycle number at which logarithmic PCR plots cross a calculated threshold line) according to the manufacturer's guidelines and the value was used to determine the Δ^{Ct} (Ct of the target gene minus the Ct of 18S ribosomal RNA controls). Relative mRNA level was calculated using the equation 2^{-ΔΔCt} as described previously (Heid et al., 1996, Genome Res. 6:986-994).

PPARα agonist Wy14643 or Fenofibrate increased gene expression of Pa9, Pa13, or Pa21 in human HuH7 cells (Table 1), primary SKMU cells (Table 2), or primary adipocytes (Table 3). Not surprisingly, gene expression patterns varied in different cell types as gene regulation is very much affected by cellular environment. These target genes Pa9, Pa13, and Pa21, were PPARα agonist-specific, because a potent PPARγ agonist, Rosiglitazone, did not significantly increase these gene expressions in human HuH7 hepatocytes at its effective concentration (Table 4).

TABLE 1
Gene expression in human HuH7 hepatocytes

| | Percentage ± SE (%) | | |
|---|---|---|---|
| Treatment | Pa9 (W38098) | Pa13 (N26311) | Pa21 (H4960) |
| Vehicle Control | 100 ± 1 | 100 ± 14 | 100 ± 37.3 |
| Wy14643, 3 uM | 90 ± 17 | 70 ± 28 | 145.5 ± 21.9 |
| Wy14643, 10 uM | 190 ± 62.1 | 100 ± 4 | 163.6 ± 35 |
| Wy14643, 30 uM | 200 ± 48 | 140 ± 26.4 | 372.7 ± 17.8 |
| Wy14643, 100 uM | 540 ± 57.8 | 130 ± 17.7 | 227.3 ± 14.8 |
| Wy14643, 300 uM | 690 ± 42.8 | 720 ± 36.4 | 481.8 ± 12.1 |
| Fenofibrate, 3 uM | 110 ± 12.7 | 180 ± 43.9 | 63.6 ± 17.3 |
| Fenofibrate, 10 uM | 90 ± 23 | 160 ± 55 | 227.3 ± 54 |
| Fenofibrate, 30 uM | 80 ± 2 | 250 ± 4.8 | 209.1 ± 20.4 |
| Fenofibrate, 100 uM | 140 ± 37.1 | 750 ± 10.8 | 300 ± 20.6 |
| Fenofibrate, 300 uM | 200 ± 11.5 | 710 ± 7.3 | 336.4 ± 24.3 |

TABLE 2
Gene expression in human primary skeletal muscle cells

| | Percentage ± SE (%) | | |
|---|---|---|---|
| Treatment | Pa9 (W38098) | Pa13 (N26311) | Pa21 (H4960) |
| Vehicle Control | 100 ± 29 | 100 ± 37.2 | 100 ± 11 |
| Wy14643, 3 uM | 110 ± 24.5 | 36.4 ± 6.4 | 130 ± 23.8 |
| Wy14643, 10 uM | 230 ± 35.7 | 45.5 ± 10 | 200 ± 48.5 |
| Wy14643, 30 uM | 870 ± 30 | 136.4 ± 38.7 | 310 ± 1.9 |
| Wy14643, 100 uM | 860 ± 0.9 | 290.9 ± 54.1 | 560 ± 16.6 |
| Wy14643, 300 uM | 610 ± 10.2 | 1672.7 ± 32 | 2250 ± 11.7 |
| Fenofibrate, 3 uM | 120 ± 34.2 | 72.7 ± 30.9 | 110 ± 14.5 |
| Fenofibrate, 10 uM | 230 ± 15.7 | 63.6 ± 11.8 | 290 ± 1.4 |
| Fenofibrate, 30 uM | 340 ± 7.4 | 181.8 ± 3 | 280 ± 52.5 |
| Fenofibrate, 100 uM | 350 ± 19.7 | 218.2 ± 2.9 | 670 ± 46 |
| Fenofibrate, 300 uM | 550 ± 14 | 309.1 ± 7.4 | 710 ± 23.1 |

TABLE 3
Gene expression in human primary adipocytes

| | Percentage ± SE (%) | | |
|---|---|---|---|
| Treatment | Pa9 (W38098) | Pa13 (N26311) | Pa21 (H4960) |
| Vehicle Control | 100 ± 17.6 | 100 ± 12 | 100 ± 30 |
| Wy14643, 10 uM | 124 ± 7.2 | 56 ± 10 | 92 ± 10 |
| Wy14643, 50 uM | 358 ± 6.9 | 55 ± 2 | 108 ± 2 |
| Wy14643, 250 uM | 437 ± 17.3 | 214 ± 7.9 | 314 ± 2.5 |
| Fenofibrate, 10 uM | 114 ± 11.4 | 70 ± 27.1 | 63 ± 5.6 |
| Fenofibrate, 50 uM | 127 ± 1.5 | 45 ± 13.3 | 109 ± 4.5 |
| Fenofibrate, 250 uM | 526 ± 25.8 | 206 ± 34.4 | 121 ± 17.3 |

TABLE 4
Gene expression in human HuH7 hepatocytes

| | Percentage ± SE (%) | | |
|---|---|---|---|
| Treatment | Pa9 (W38098) | Pa13 (N26311) | Pa21 (H4960) |
| Vehicle Control | 100 ± 22.3 | 100 ± 25.2 | 100 ± 8 |
| Rosiglitazone, 1 μM | 147 ± 31.2 | 217 ± 20.2 | 97 ± 17.5 |

### EXAMPLE 2

### Bioinformatic Characterization of Genes PA9, 13 and 21

Bioinformatic programs sponsored by the National Center for Biotechnology Information (NCBI; http://www.ncbi.nlm.nih.gov) were used in the analysis of DNA sequences. The GenBank database (http://www.ncbi.nlm.nih.gov/Genbank/index.html) was used to retrieve DNA sequences. The SwissPro database (http://www.ebi.ac.uk/swissprot) was used to retrieve protein sequences. The BLAST program (http://www.ncbi.nlm.nih.gov/BLAST) was used for gene search. The Motif program (http://motif.genome.ad.jp,) was used for searching functional DNA structures (motifs, regulatory sequences, domains, etc.). The LocusLink program (http://www.ncbi.nlm.nih.gov/LocusLink) was used for searching sequence and descriptive information about genetic loci. In addition, the Wisconsin Package (GCG, Genetics Computer Group) was used for sequence editing and sequence assembly (SEQED), sequence comparisons (GAP, BESTFIT, PILEUP), and DNA sequence translation (TRANSLATION).

Pa9

The original annotation for the Pa9 gene sequence used on the microarray was GenBank accession number W30988: Homo sapiens cDNA clone similar to human fibrinogen-related protein HFREP-1 precursor. BLAST search indicated that the sequence of W30988 (with multiple “n” in the sequence due to sequencing error) shared about 97% (339/349; the score of comparison is e-162) sequence identity to several GenBank sequence entries, such as GenBank accession Nos: BC023647, AF202636, AF169312, AF153606, AB056477, E39784 BD075801, AX578037, AX464136, AX278133, AX201322, AX079971, AX079874, AX068560, AR243163, and AR194215. These GenBank sequence entries appeared to be identical and encode a protein of about 406 amino acid residues (SEQ ID NO:2). The encoded protein was claimed as a human NL2 TIE ligand homologue polypeptide in U.S. Pat. No. 6,348,350, and as a human Fibrinogen Domain Related (FDRG) protein in WO0177151. Other annotations for the protein include angiopoietin-like protein PP1158, and TIE receptor tyrosine kinase ligand homolog, etc.

WO0177151 (Example 12) disclosed that murine FDRG expression level increased 10 fold in NIH3T3 fibroblasts stably expressing PPARγ after the cells were treated with 10 μg/ml pioglitazone (an agonist for PPARγ) for 2 hours. In addition, WO0177151 (Example 13) disclosed that murine FDRG expression in Zucker Diabetic Fatty Rats was upregulated 3-5 fold in white adipose tissue (WAT) when the animal was treated with chronic troglitazone (an agonist for PPARγ). These observations are compatible with FDRG being a target gene of PPARγ in rodents.

In addition, Kersten et al (2000, J. Biolo. Chem. 275: 28488-493) showed that expression of murine FIAF (fasting induced adipose factor), which is identical to the murine FDRG, was decreased in livers of PPARα null mice. They also found that expression of murine FIAF was increased by WY14643 treatment in PPARα wild type mouse liver cells but not in PPARα null mouse liver cells. In addition, they showed that expression of murine FIAF was decreased in WAT of heterozygous PPARγ mutant mice. These results are compatible with FDRG being a target gene of both PPARα (in liver) and PPARγ (in WAT) in rodents.

However, one could not readily conclude that FDRG is a target gene for either PPARα or PPARγ in a human simply because it is a target gene for PPARα or PPARγ in a rodent. Action of fibrate or other PPARα agonist is species specific and many genes induced by fibrates or other PPARα agonists in rodents are not induced in humans. In addition, because the mouse FDRG shares relatively low sequence identity with the human FDRG: about 73% sequence identity at the cDNA level and about 76% sequence identity at the protein level, the mechanism of gene regulation for FDRG in mouse and human may vary.

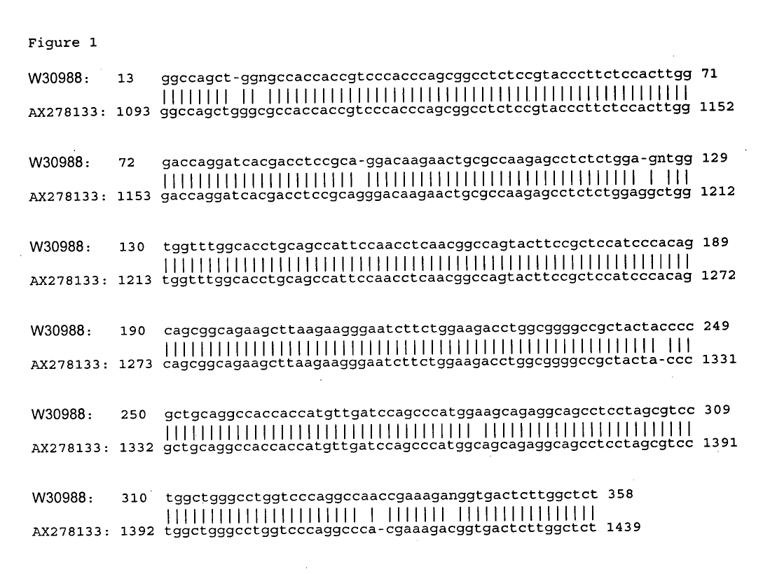

The nucleotide sequence of the human FDRG gene (AX278133) is provided in SEQ ID NO: 1 and the sequence alignment between W30988 and FDRG gene is shown in FIG. 1. Examination of the sequence alignment suggested that sequence discrepancies between W30988 and the human FDRG gene are mostly due to sequencing errors in W30988. Quantitative real-time RT-PCR was performed to test whether gene expression of the human FDRG gene is regulated by PPARα. The expression level of gene Pa9 and gene FDRG were measured from human skeletal muscle cells stimulated with 30 μM or 100 μM Wy14643 using RT-PCR experiments similar to those described in Example 1. PCR primers and probes used for measuring Pa9 gene expression were SEQ ID NO: 7, 8 and 9 as described in Example 1. These primers and probes can hybridize with W30988 under stringent hybridization condition. PCR primers and the fluorescent probe used for measuring FDRG gene expression were designed such that they can hybridize with the FDRG gene outside the region that shares sequence homology with the nucleotide sequence of W30988. The primers and probe for FDRG gene are: SEQ ID NO: 16, 5′-AGCCCCCTTT CTGAGTGCA, SEQ ID NO: 17, 5′-CCCTTGGTCC ACGCCTCTA, and SEQ ID NO: 18, 6FAM-TGAGATCGAG GCTGCAGGAT ATGCTCA. As shown in Tale 5, Wy14643 induced the Pa9 gene expression by 3.64-fold at the concentration of 30 μM and 6.45-fold at the concentration of 100 μM. Measurement from the same RNA sample indicated that, Wy14643 induced the FDRG gene expression by 4.37-fold at the concentration of 30 μM and 7.8-fold at the concentration of 100 μM. Similar results were obtained from a different set of human SKM RNA samples (data not shown). Based on sequence homology between the Pa9 gene (W30988) and the FDRG gene (NM_{—}139314), and the mRNA quantification data described herein, it is reasonable to conclude that: 1) W30988 is actually a partial sequence of the full-length cDNA sequence of NM_{—}139314; 2) the human Pa9 gene comprising the sequence of W30988 is identical to the human FDRG gene; and 3) the human FDRG gene is indeed up-regulated by PPARα.

TABLE 5
Gene expression in human primary skeletal muscle cells

| | Pa9 gene expression | | FDRG gene expression | |
|---|---|---|---|---|
| Treatment | Fold of Induction | SE | Fold of Induction | SE |
| Veh | 1.00 | 0.20 | 1.00 | 0.14 |
| WY14643, 30 uM | 3.64 | 0.01 | 4.37 | 0.06 |
| WY14643, 100 uM | 6.45 | 0.18 | 7.80 | 0.79 |

2) Pa13

The original annotation for the Pa13 gene sequence used on the microarray was GenBank accession number N26311, a cDNA clone with no other functional annotation. BLAST search indicated that nucleotide sequence of N26311 shared about 98% (416/424) sequence identity to multiple suspected cancer cell gene clones disclosed in WO194629, for example, GenBank accession No AX329948. In addition, N26311 shared about 95% (387/404) identity to several other GenBank sequence entries, including GenBank accession numbers, U88323, AF019770, AB000584, E09952, AX468429, AX408886, AR236374, AR127403, BC000529, AX052609, AR091376, E10038, and AR107279. These GenBank sequence entries appeared to be identical and encode a protein of about 308 amino acid residues (SEQ ID NO: 4). GenBank annotations for this encoded protein include TGF-beta superfamily protein, placental bone morphogenic protein PLAB, macrophage inhibitory cytokine-1 (MIC-1), TGF-beta superfamily protein, non-steroidal anti-inflammatory drug activated gene with anti-tumorigenic properties, gene expressed in liver cancer, and prostatic growth factor, etc. No correlation between the protein and diabetes (or obesity, or hyperlipidemia) has been established or described previously.

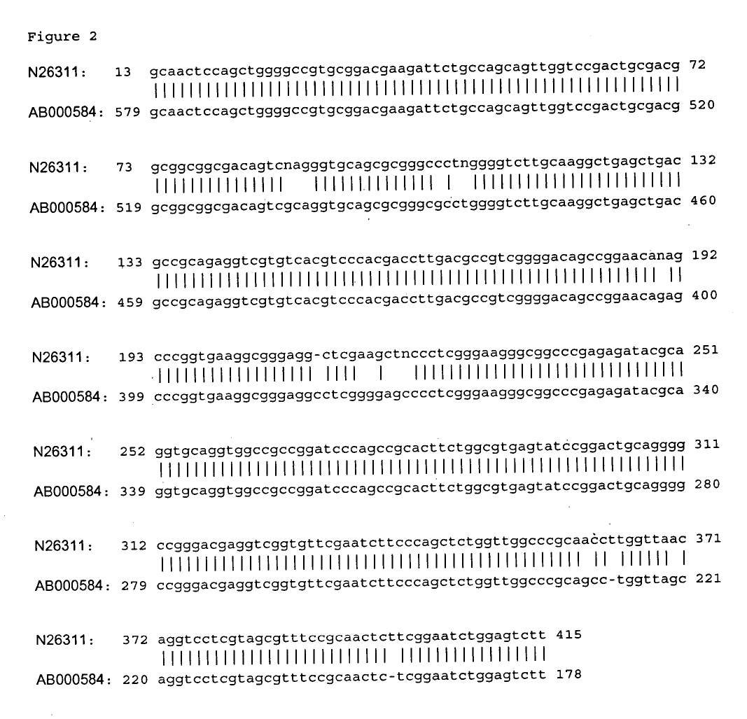

The nucleotide sequence of a representative of these GenBank entries, AB000584, is listed in SEQ ID NO: 3, and the sequence alignment between N26311 and AB000584 is shown in FIG. 2. Examination of the sequence alignment suggested that the sequence discrepancies between N26311 and AB000584 are mostly due to sequencing errors in N26311. Therefore, the human Pa13 gene, which comprises the nucleotide sequence of N26311, is likely to be identical to the TGF-beta superfamily protein gene depicted in AB000584 (Yokoyama-Kobayashi et al., 1997, J Biochem (Tokyo). 122(3): 622-6). Indeed, it was found that the human TGF-beta superfamily protein gene was up-regulated by PPARα, using PCR primers and probe (SEQ ID NO: 10, 11, and 12) that hybridize with the TGF-beta superfamily protein gene outside the region that shares sequence homology with the nucleotide sequence of N26311 (see data in Example 1).

In addition, potential PPREs were identified in the 5′ untranslated region (5′-UTR) of the human Pa13 gene. SEQ ID NO: 19 shows the 5′-UTR, from −2538 to −1 bp immediately up-stream of the ATG translation initiation site of the TGF-beta superfamily protein gene, which is identical to Pa13 gene. Nucleotide sequence of the 5′-UTR was downloaded from the human genome sequence database, from 152956 bp to 155493 bp of GenBank Accession No: AC008397. Two potential transcription start sites were identified at base 970 and 2508 of SEQ ID NO: 19. Three potential PPREs each comprising the sequence of AGGTCA were identified at base 160, 564, and 1033 of SEQ ID NO: 19. Several other potential PPRE sites having the sequences of TGACCT, AGGTCG, or GGGTCA have also been found within SEQ ID NO: 19. This result is consistent with that the Pa13 gene is transcriptionally regulated by PPAR in human cells.

3) Pa21:

The original annotation for the Pa21 gene sequence used on the microarray was GenBank accession number H49601: a cDNA clone similar to anaphylatoxin chemotactic receptor. BLAST search indicated that nucleotide sequence of H49601 shared about 93% (384/410) sequence identity to several GenBank sequence entries, including GenBank accession numbers, AF075053, AL833944, AL121758, BC017001, AK098418, and AX368959. These GenBank sequence entries appeared to be identical. The annotations for most of the GenBank entries are cDNA clones with unknown functions. Some of the annotations are suspected cancer cell gene clones. H49601 also shares 93% (384/410) sequence identity to the complemental sequence of GenBank accession number NM_{—}080725 (SEQ ID NO: 5), which encodes a protein of 137 amino acid residue (SEQ ID NO: 6). The protein was named chromosome 20 open reading frame 139 (C20orf139) previously. No correlation between C20orf139 and diabetes (or obesity, or hyperlipidemia) has been established or described previously.

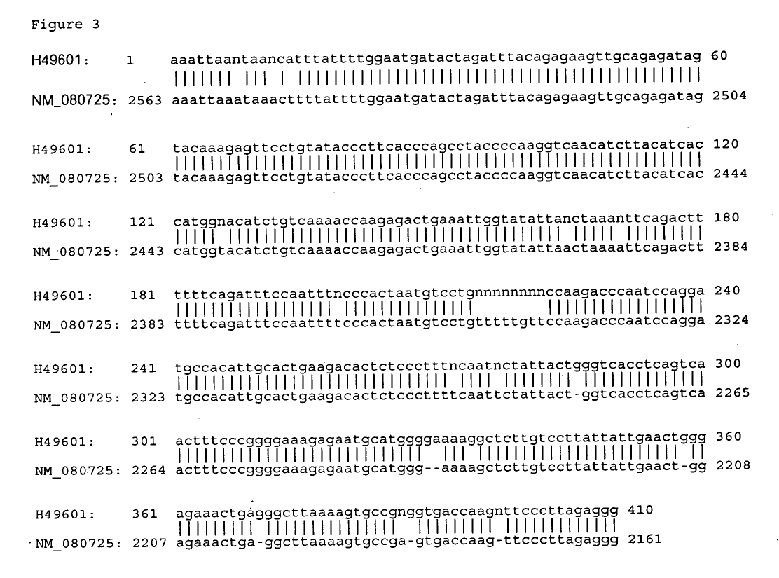

The sequence alignment between H49601 and NM_{—}080725 is shown in FIG. 3. Examination of the sequence alignment suggested that the sequence discrepancies between H49601 and NM_{—}080725 are mostly due to sequencing errors in H49601. Therefore, the Pa21 gene, which comprises the nucleotide sequence of H49601, is likely to be identical to the C20orf139 gene depicted in NM_{—}080725. Indeed, it was found that the human C20orf139 gene was up-regulated by PPARα, using PCR primers and probe (SEQ ID NO: 13, 14, and 15) that hybridize with the C20orf139 gene outside the region that shares sequence homology with the nucleotide sequence of H49601 (see data in Example 1).

In addition, potential PPREs were identified in the 5′-untranslated region (5′-UTR) of the C20orf139 gene. SEQ ID NO: 20 shows the 5′-UTR, from −1 bp to −3000 bp immediately up-stream of the ATG translation initiation site of the C20orf139 gene, which is identical to the Pa21 gene. Nucleotide sequence of the 5′-UTR was downloaded from the human genome sequence database, 568887bp to 571886 bp of GenBank accession number Hs20_{—}11544. Two “AGGTCA” PPRE sites were found at −2201 bp and −2866 bp, respectively. Three “TGACCT” sites were found at −872 bp, −893 bp, −2344 bp, respectively. This result is consistent with that the C20orf139 is transcriptionally regulated by PPAR in human cells.

### EXAMPLE 3

### Reduced PPARα Resulted in Reduced Induction of Gene Expression of Pa9, 13 or 21

Human SKM cells with reduced PPARα expression was first constructed using siRNA technique. Gene expression of Pa9, 13, and 21 in these cells was then measured to confirm that these genes were indeed regulated by PPARα.

SiRNA oligos specific to PPARα were designed and used to knock-down or reduce the expression level of PPARα in human SKMU cells following procedures known to those skilled in the art (Brummelkamp et al., 2002, Science, 296: 550-553). Four sets of siRNA oligo were originally designed using GenBank accession number S74349 (human peroxisome proliferator activated receptor alpha, cDNA; PPARα) as the template, and the software, Target Finder (Ambion, Austin, Tex.). Among the four sets of oligos, two sets were found to be more efficient and were chosen for PPARα-knockdown studies. The sequences for the two sets of siRNA correspond to 907 bp to 927 bp (SEQ ID NO: 21, 5′-AACGATCAAG TGACATTGCT A) and 1106 bp to 1126 bp of the cDNA (SEQ ID NO: 22, 5′-AACTGGATGA CAGTGATATC T) relative to the first nucleotide of the start codon. The oligos were chemically synthesized by Ambion Inc. (Austin, Tex.). The universal negative control siRNA oligo was purchased from Ambion.

Human primary skeletal muscle cells (hSKM, provided by Cambrex, Md.) were prepared the day before transfection at the density of 4.67×10⁴ cells/per 60 mm dish (about 20% confluence). The cells were cultured at 37° C., 5% CO₂ for 24 hrs with antibiotics-free SKGM medium (Cambrex, Md.).

Transfection of siRNAs into the cultured cells was carried out following the manufacturer's instruction. Briefly, before the transfection, siPORT Amine (Ambion, Tex.) was first diluted with SKGM medium free of antibiotics and serum (10 μl siPORT/ml), followed by adding siRNA (50 nM) into the mixture. SKM cells (prepared in advance in multiple dishes) were transfected with 2 ml of the mixture for 4 hrs. Additional 8 ml of fresh SKGM medium (without antibiotics) were added into each dish to maximize cell growth and reduce potential cytotoxicity caused by the transfection reagent. As controls, the cells were also transfected with siPORT Amine alone or siPORT Amine plus universal negative control siRNA.

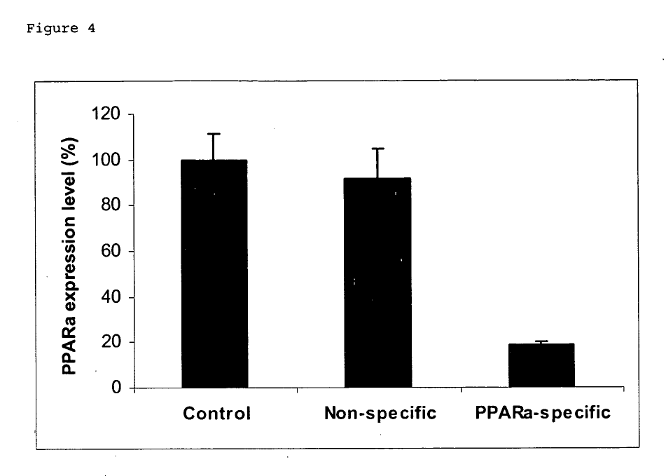

Total RNA was isolated from some of the transfected dishes after 48 hrs of transfection using a modified Qiagen RNeasy method (Qiagen, Calif.). Real time PCR was used to validate that the PPARα expression level had been truly knocked down by siRNA. It was found that under optimal conditions used, the expression level of PPARα was reduced by 70-80% in SKM cells by siRNA (FIG. 4).

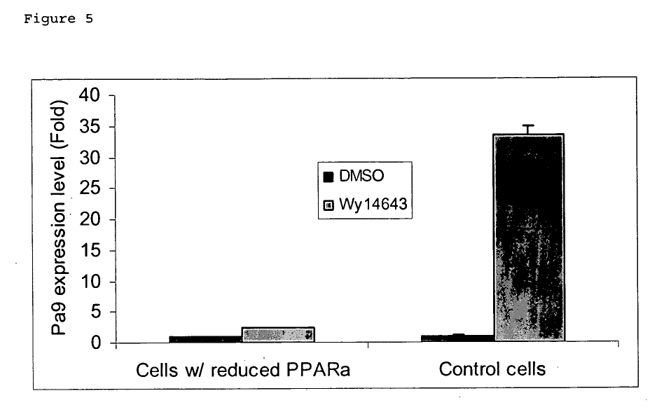

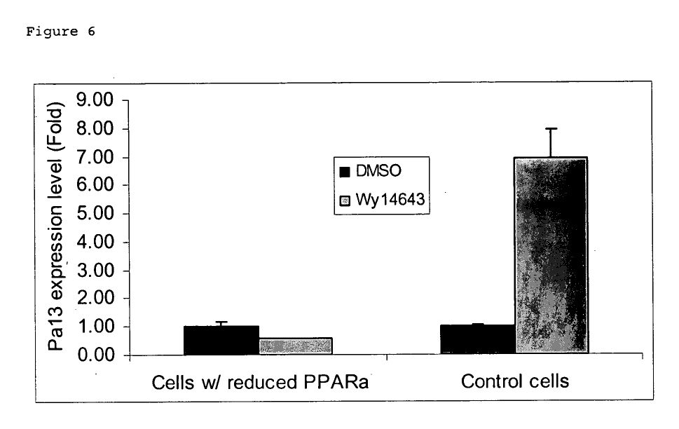

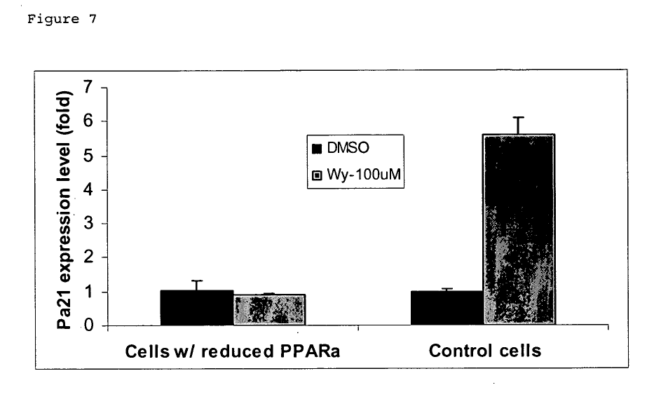

After the validation, the PPARα-knocked-down cells were treated with different compounds for an additional 16 hrs. Again, total RNA was isolated from the cells and real-time PCR was performed and the expression levels of target genes of interest, such as Pa9, 13 and 21, were quantitatively measured using the RT-PCR technique described in Example 1. The results showed that: A) In SKM cells with reduced gene expression of PPARα, gene expression of Pa9 was only increased 2.35-fold upon Wy14643 treatment, as compared to 33.48-fold induction in cells without reduced expression level of PPARα (FIG. 5); B) similar results were observed for the genes of Pa13 (FIG. 6) and Pa21 (FIG. 7); and C) In SKM cells with reduced gene expression of PPARα, the expression level of a known non- PPARα target gene, PPARd, was not altered.

As apparent from the foregoing, the invention discovered for the first time that the human Pa9 gene, Pa13 gene, and Pa21 gene defined herein are target genes of peroxisome proliferator-activated receptor alpha (PPARα). While the above detailed description and preferred embodiments have been provided to illustrate the invention and its various features and advantages, it will be understood that invention is defined not by the foregoing, but by the following claims as properly construed under principles of patent law.

1. A method of determining the activity of PPARα in a biological sample from a subject, comprising the step of determining the expression level of a gene selected from the group consisting of a human Pa9 gene, a Pa13 gene, and a Pa21 gene in the sample.

2. A method of evaluating the effectiveness of a treatment for a metabolic abnormality in a subject comprising the steps of:
a. determining the expression level of a gene selected from the group consisting of a human Pa9 gene, a Pa13, and a Pa21 in the subject during or after the treatment; and
b. comparing the expression level determined in step a) with the expression level of the gene in the subject prior to the treatment;
wherein an increase in the expression level of the gene in the subject during or after the treatment indicates that the treatment for the metabolic abnormality in the subject is effective.

3. The method of claim 2, wherein the metabolic abnormality is dyslipidaernia or a condition associated with dyslipidaemia.

4. The method of claim 2, wherein the metabolic abnormality is atherosclerosis, obesity, thrombosis or coronary artery disease, hypertension, angina, chronic renal failure, peripheral vascular disease, stroke, type II diabetes, or metabolic syndrome (syndrome X).

5. The method of claim 2, wherein the treatment for a metabolic abnormality involves an agent capable of regulating the biological activity of peroxisome proliferator-activated receptor alpha (PPARα).

6. The method of claim 5, wherein the agent is a PPARα agonist.

7. The method of claim 2, further comprising the step of obtaining a biological sample from the subject, wherein the expression level of the gene in the subject is determined from the biological sample.

8. The method of claim 7, wherein the biological sample from the subject is peripheral blood mononuclear cells.

9. The method of claim 7, wherein the biological sample from the subject is from adipose tissue, liver, heart, kidney or muscle.

10. The method of claim 2, wherein the expression level of the gene is determined by measuring the amount of mRNA of the gene in the subject.

11. The method of claim 10, wherein a nucleic acid probe capable of hybridizing to SEQ ID NO: 1 under stringent hybridization conditions is used to measure the amount of human Pa9 mRNA in the subject.

12. The method of claim 10, wherein a nucleic acid probe capable of hybridizing to SEQ ID NO: 3 under stringent hybridization conditions is used to measure the amount of Pa13 mRNA in the subject.

13. The method of claim 10, wherein a nucleic acid probe capable of hybridizing to SEQ ID NO: 5 under stringent hybridization conditions is used to measure the amount of Pa21 mRNA in the subject.

14. The method of claim 2, wherein the expression level of the gene is determined by measuring the amount of polypeptide encoded by the gene in the subject.

15. The method of claim 14, wherein an antibody capable of binding specifically to SEQ ID NO: 2 is used to measure the amount of human Pa9 protein in the subject.

16. The method of claim 14, wherein an antibody capable of binding specifically to SEQ ID NO: 4 is used to measure the amount of Pa13 protein in the subject.

17. The method of claim 14, wherein an antibody capable of binding specifically to SEQ ID NO: 6 is used to measure the amount of Pa21 protein in the subject.

18. A kit for evaluating the effectiveness of a treatment for a metabolic abnormality in a subject, comprising a nucleic acid probe that hybridizes under stringent hybridization condition to a nucleic acid molecule selected from the group consisting of SEQ ID NO: 1, SEQ ID NO: 3, and SEQ ID NO: 5, or a complement thereof,

19. A kit for evaluating the effectiveness of a treatment for a metabolic abnormality in a subject, comprising an antibody that binds specifically to a polypeptide molecule selected from the group consisting of SEQ ID NO: 2, SEQ ID NO: 4, and SEQ ID NO: 6.

20. The kit of claim 18 and 19 further comprising instructions for determining whether a treatment for a metabolic abnormality in a subject is effective or not.

21. A method of identifying a compound useful for treating a metabolic abnormality in a subject,, comprising the steps of:
a. contacting a polypeptide encoded by a human Pa9 gene, a Pa13 gene, or a Pa21 gene with a solution comprising a buffer and a candidate or test compound;
b. measuring the effect of the test compound on the activity of the polypeptide; and
c. comparing the result of step (b) with that of a control wherein the polypeptide is contacted with only the buffer.

22. The method of claim 21, wherein the polypeptide is expressed from a host cell.

23. The method of claim 21, wherein the polypeptide is associated with an isolated membrane preparation.

24. The method of claim 21, wherein the polypeptide is purified.

25. A method of identifying a compound that is useful for treating a metabolic abnormality in a subject, comprising the steps of:
a. contacting a PPARα-responsive system with a solution comprising a buffer and a candidate or test compound;
b. measuring from the PPARα-responsive system the expression level of a gene controlled by a regulatory sequence of a human Pa9 gene, a Pa13 gene, or a Pa21 gene; and
c. comparing the result of step (b) with that of a control lacking the test compound.

26. The method of claim 25, wherein the PPARα-responsive system is an animal, a tissue, or a cell.

27. The method of claim 25 wherein the PPARα-responsive system comprises a functional PPARα protein and a gene controlled by a regulatory sequence of a human Pa9 gene, a Pa13 gene, or a Pa21 gene.

28. The method of claim 27, wherein the functional PPARα protein is expressed endogenously from the PPARα-responsive system.

29. The method of claim 27, wherein the functional PPARα protein is expressed recombinantly from an exogenously DNA molecule introduced into the PPARα-responsive system.

30. The method of claim 27, wherein the gene controlled by a regulatory sequence of a human Pa9 gene, a Pa13 gene, or a Pa21 gene is the human Pa9 gene, the Pa13 gene, or the Pa21 gene, respectively.

31. The method of claim 27, wherein the gene controlled by a regulatory sequence of a human Pa9 gene, a Pa13 gene, or a Pa21 gene is a reporter gene.

32. The method of claim 31, wherein the reporter gene is selected from the group consisting of genes of green fluorescent protein (GFP), β-galactosidase (lacZ), luciferase (luc), chloramphenicol acetyltransferase (cat), β-glucuronidase, neomycin phosphotransferase, and guanine xanthine phosphoribosyl-transferase.

33. A PPARα-responsive system comprising a functional PPARα protein and a nucleic acid molecule comprising the coding sequence of a reporter gene operably linked to the regulatory sequence of a human Pa9 gene, a Pa13 gene, or a Pa21 gene.

34. An isolated nucleic acid molecule comprising the coding sequence of a reporter gene operably linked to the regulatory sequence of a human Pa9 gene, a Pa13 gene, or a Pa21 gene.

35. The isolated nucleic acid molecule of claim 34, wherein the reporter gene is selected from the group consisting of genes of green fluorescent protein (GFP), β-galactosidase (lacZ), luciferase (luc), chloramphenicol acetyltransferase (cat), β-glucuronidase, neomycin phosphotransferase, and guanine xanthine phosphoribosyl-transferase.

36. A method of treating a metabolic abnormality in a subject comprising the step of administering to the subject a therapeutically effective amount of a composition that alters the gene expression or biological activity of a human Pa9 gene, a Pa13 gene, or a Pa21 gene in cells from a subject, wherein the composition is not a known PPARα agonist or antagonist.

37. The method of claim 36, wherein the composition is a siRNA that targets the mRNA of a human Pa9 gene, a Pa 13 gene, or a Pa21 gene, for degradation in a cell of the subject.

38. The method of claim 36, wherein the composition is a nucleic acid molecule capable of expressing a human Pa9 gene, a Pa13 gene, or a Pa21 gene in a cell of the subject.
